(19) United States
(12) Patent Application Publication
(10) Pub. No.: US 2026/0265825 A1
(43) Pub. Date: Sep. 10, 2026

(54) MULTI-OMIC PROFILING SIGNATURES FOR COVID-19

(71) Applicant: Verily Life Sciences LLC, Dallas, TX (US)

(72) Inventors: Katherine DRAKE, Boulder, CO (US); Samuel KATZ, San Francisco, CA (US); Charles KIM, San Bruno, CA (US); Jacqueline LEUNG, Daly City, CA (US); Jack LIN, San Bruno, CA (US); Gary TONG, Berkeley, CA (US); Benjamin YUEN, San Mateo, CA (US)

(21) Appl. No.: 19/167,290

(22) PCT Filed: Apr. 18, 2024
(86) PCT No.: PCT/US2024/025128

§ 371 Date: Sep. 19, 2025

Related U.S. Application Data
(60) Provisional application No. 63/496,918, filed on Apr. 18, 2023.

(51) Int. Cl. C12Q 1/6883; G01N 33/68

(52) U.S. Cl. CPC C12Q 1/6883; G01N 33/6893; C12Q 2600/118; C12Q 2600/158; G01N 2333/165; G01N 2800/50

(57) ABSTRACT

A signature for identifying a subject at risk of developing a severe reaction to a SARS-CoV virus by detecting one or more of elevated serum cytokines, reduced monocyte subclasses, differentially expressed genes in monocyte subclasses, differentially expressed gene in CD8+ effector memory T cells, and elevated chromatin accessibility in intermediate monocytes are disclosed herein. Also disclosed are methods for obtaining the signature, methods of identifying a subject at risk of developing a severe reaction to a SARS-CoV virus, and methods of treating COVID-19.

### CROSS-REFERENCE TO RELATED APPLICATION

This application claims the benefit of U.S. Provisional Patent Application 63/496,918, filed on Apr. 18, 2023; the contents of which is incorporated by reference in its entirety.

### TECHNICAL FIELD

This disclosure relates generally to a multi-omic profiling signature for identifying subjects at risk of developing a severe reaction to a SARS-CoV virus, such as COVID-19, methods of generating the same, and methods of treating a subject at risk of developing a severe reaction to a SARS-CoV virus.

### BACKGROUND

Since the beginnings of the coronavirus disease of 2019 (COVID-19) pandemic, the immune response has been studied in patients from the standpoint of both the host antiviral response and unchecked inflammatory pathology. While this has resulted in the rapid development of testing, prophylactic, and interventional methods for managing the spread and severity of SARS-CoV-2 infection, the health burden of resultant COVID-19 remains high. Global excess deaths from the period of January 2020 to December 2021 were estimated to be 14.83 million, with 20 countries accounting for over 80% of deaths. Yet even at the beginning of 2023, daily 7-day rolling averages of excess deaths due to COVID-19 were still 350+ in the United States and 1,850+ worldwide. If this pace continues without abatement, mortality from COVID-19 in the United States will be on track to continue to exceed that of both influenza and respiratory syncytial virus, the two most common causes of respiratory infection-associated deaths before the pandemic, combined. However, it has also long been recognized that the majority of individuals infected with SARS-CoV-2 develop a relatively mild, self-resolving COVID-19 disease not requiring advanced medical intervention, and it is only in a minority of patients that a severe disease course occurs requiring hospitalization and costly interventions.

This heterogeneity, along with continuing mortality, highlights the need for techniques to differentiate between high-risk patients and low-risk patients.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show an overview of the Immune Profiler cell frequency and subset-resolved multi-omic data. FIG. 1A shows cell subset frequencies grouped by Non-progressor versus Progressor pairwise outcome group box plots. FIG. 1B shows the number of significant differentially expressed genes (DEGs) between Non-progressors and Progressors by cell subset from RNA-seq data. FDR cutoffs for DEGs were set and graphed as ≤0.05 (black bar) or ≤0.1 (gray bar). FIG. 1C shows the number of differentially expressed proteins (DEPs) between Non-progressors and Progressors by cell subset from TaPE-seq data. FDR cutoffs for DEPs were set and graphed as ≤0.05 (black bar) or ≤0.1 (gray bar). FIG. 1D shows the number of differentially accessible regions (DARs) between Non-progressors and Progressors by cell subset from ATAC-seq data. FDR cutoffs for DARs were set and graphed as ≤0.05 (black bar) or ≤0.1 (gray bar).

FIGS. 2A-2C show elevated cytokine and chemokine levels in Progressors. FIG. 2A shows the concentration in pg/ml of cytokines and chemokines elevated in plasma of Progressors compared to Non-progressors with a FDR≤0.05 as measured by Luminex assay. FIG. 2B shows cytokine concentration ratios for Non-progressors and Progressors. interleukin-6 (IL-6); interferon-alpha2 (IFNα2); interferon-gamma (IFNγ); tumor necrosis factor-alpha (TNFα); interleukin-17A (IL-17A); interleukin-10 (IL-10). FIG. 2C shows Spearman's correlations between immune cell subset frequencies and plasma IL-6 concentrations with a ρ≥0.2 or ρ≤−0.2.

FIG. 3A-3E show perturbations in Progressor monocyte cell subset frequencies. FIG. 3A shows the ratio of distinct monocyte subsets to total frequency of monocytes. Significance assessed by p-values for each individual test. FIG. 3B shows Principal Component Analysis (PCA) of all participant monocyte subsets ATAC-seq data showing a spectrum, left panel, ranging from classical monocytes (red), to intermediate monocytes (green), to non-classical monocytes (blue). Right panel, the intermediate monocytes of Progressors (blue) or Non-progressors (red) are colored. FIG. 3C shows a boxplot of Progressor and Non-progressors intermediate monocytes on the ATAC-Seq epigenetic spectrum. A Wilcoxon signed-rank test was performed to measure significance of Progressor versus Non-progressor intermediate monocyte differences in their distances between the classical monocyte centroid and the non-classical monocyte centroid. FIG. 3D shows a typical biaxial CD14/CD16 plot gating methods focus on defined gates for monocytes that delineate classical, intermediate, and non-classical cell subsets (left). Finer partitions placed along the biaxial CD14/CD16 plot, from G0-G10, allows for comparative measurements of frequency across the MoCl—MoIn—MoNC transitional path (right). FIG. 3E shows G0-G10 gating partition cell frequencies of total monocytes in Progressors and Non-progressors. The MoCl (G0-G1), MoIn (G6-G7), and MoNC (G9-G10) labels indicate partitions that would typically be ascribed to classical, intermediate, and non-classical monocyte CD14/CD16 gates.

FIGS. 4A-4C show monocyte Progressor versus Non-progressor DEGs. FIG. 4A shows DEGs between Progressors versus Non-progressor monocyte cell subsets (at FDR≤0.1). FIG. 4B shows DEGs with proximally associated DARs comparing all Progressor to Non-progressor cell subsets (at FDR≤0.1). Distance indicates the distance between the ATAC peak from the corresponding gene's TSS (transcriptional start site). FIG. 4C shows chromatin contact via HiChIP between regions with eQTL SNPs and the THBD TSS in monocyte subsets. The dark vertical line represents the transcriptional start site. Lines for rs6083011, rs6113954, and rs6515302 are overlapping in the figure. The y-axis is expressed as counts per million (CPM).

FIGS. 5A-5E show that CD8+ effector memory T (T8em) cells display a highly activated phenotype in Progressors. FIG. 5A shows the mean log 2 fold-change (log 2FC) of Progressor T8em relative to Non-progressor T8em for activation-related genes by RNA-seq. GSEA was used to determine pathway enrichment scores. FIG. 5B shows the log 2 fold-change (log 2FC) in chromatin accessibility by ATAC-seq of Progressor T8em relative to Non-progressor T8em for hyperactivation-related genes (bars colored by gene). Multiple bars indicate multiple identified peaks within +/−1 kb of gene TSS. FIG. 5C shows mean log 2 fold-change (log 2FC) of T cell dysfunction or activation-related genes in Progressor T8em relative to Non-progressor T8em by RNA-seq. GSEA was used to determine pathway enrichment scores. FIG. 5D shows log 2 fold-change (log 2FC) in chromatin accessibility by ATAC-seq of Progressor T8em relative to Non-progressor T8em for dysfunction-related genes. Multiple bars indicate multiple identified peaks within +/−1 kb of gene TSS. FIG. 5E shows log 2 fold-change (log 2FC) of cell surface protein levels by TaPE-seq in Progressor T8em relative to Non-progressor T8em for regulatory and activation markers. GSEA was used to determine pathway enrichment scores.

FIG. 6 shows that the 24 immune cell subsets resolved by fluorescent surface marker staining and sorting represent canonically defined subsets with respect to the ability to immunophenotype by cell surface markers, and known characteristics of lineage and functional differentiation.

FIG. 7 shows the viral load from mid-turbinate swabs early in disease course, as measured by detection of the viral genes N and ORF1a by ddPCR or in the IgG antibody reactivity towards SARS-CoV-2 S and N protein or other related human coronaviruses. RPP30 is the positive control.

FIG. 8 shows the cell subset frequency correlation heatmaps. Heatmaps show pairwise spearman correlations within Non-progressor and Progressor cohorts. Green, red, and magenta boxes indicate correlated clusters of myeloid cell, B cell, and effector T cell subsets, respectively.

FIG. 9A-9D shows molecular pathway analysis and machine learning modeling. FIG. 9A shows GSEA pathway analysis comparing Non-progressors and Progressors by cell subset from RNA-seq data. FIG. 9B shows GSVA pathway analysis comparing Non-progressors and Progressors by cell subset from RNA-seq data. FIG. 9C shows the proportion of DARs within each subset (at FDR≤0.1) found within each annotated genomic region. The total number of DARs discovered is indicated above each bar. FIG. 9D shows block sparse partial least squares-discriminant analysis performed for each cell subset using RNA-seq, ATAC-seq, and progression outcome data assessed the classification performance for each subset. Cell subsets were ranked in their performance by the area under the ROC curve generated from Leave-one-out cross-validation (LOOCV).

FIG. 10 shows plasma cytokines and chemokines that are elevated in Progressors. Cytokines and chemokines elevated in plasma of Progressors compared to Non-progressors with a FDR≤0.1 as measured by Luminex assay. Comparative values are expressed as log 10 fold change (log 10FC).

FIG. 11 shows RNA gene expression levels of IL-6, IFNγ, and TNF in Non-progressors and Progressors. IL6, IFNγ, and TNF gene expression levels are displayed as transcripts per million (TPM) by cell subset.

FIG. 12 shows pathway analysis of combined monocyte DEGs. Analysis was done for the Wikipathways and Reactome pathway sets (curated by the MsigDB collections) using a hypergeometric test of all monocyte DEGS with a 0.1 FDR cutoff.

FIG. 13 shows an example chromatic contact map vit HiChIP around THBD in all subsets. The dark vertical line represents the transcriptional start site, and colored vertical lines represent variant positions.

FIG. 14A-14B shows univariate differential gene expression analysis in T8em. FIG. 14A shows log 2 fold-change (log 2FC) of DEGs in Progressor T8em relative to Non-progressor T8em at FDR<0.05. FIG. 14B shows DEGs in Progressor T8em detected at FDR<0.05 overlapping with mouse gene equivalents in an LCMV model of T cell activation.

FIG. 15 illustrates a network environment that includes an Immune Profiler platform that is executed by a computing device.

FIG. 16 illustrates an example of a computing device that is able to implement an Immune Profiler platform.

FIG. 17 illustrates exemplary training process in which multi-omic data is used by a machine learning algorithm to create a machine learnt model, as well as an inferencing process in which the machine learnt model is applied to better understand the likelihood that a subject will experience severe symptoms from the SARS-CoV virus.

### DETAILED DESCRIPTION

Embodiments of a multi-omic profiling signature for identifying subjects at risk of developing a severe reaction to a SARS-CoV virus, such as COVID-19; methods of generating the multi-omic profiling signature; and methods of treating a subject based on the multi-omic profiling signature are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Since the mechanism behind the difference in disease progression between high-risk COVID-19 subjects and low-risk COVID-19 subjects is not yet well understood, it is essential to study immune responses to COVID-19 within patients as they develop. Such multi-omic studies have focused on the mechanisms and signatures of severe disease at later phases.

The present technology surveys the immune response from patient biospecimens collected during the early phase of infection using multi-omic assays. Early immune response signatures in participants classified are compared to Progressor and Non-progressor outcome groups to find mechanisms that distinguish those on higher risk trajectories from milder disease. The present technology fills the knowledge gap by highlighting early immune observations through the comparative lens of eventual Progressor versus Non-progressor clinical outcome. As such, the present technology includes a signature for determining whether a subject who has tested positive for a SARS-CoV virus, such as COVID-19 is a progressor or a non-progressor.

### Definitions

As used herein, a “signature” or “biomarker” includes a set of cellular and/or molecular information that can be used to identify an event. In some embodiments, the event identified by a signature may be the probability, possibility, likelihood, or risk of developing severe disease, such as a severe reaction to a SARS-CoV virus. In some embodiments, the cellular and/or molecular information of the signature may include genome, proteome, transcriptome, epigenome, and/or microbiome information. As used herein, the “multi-ome” or “multi-omic” information includes more than one of genome, proteome, transcriptome, epigenome, and/or microbiome information.

As used herein, a “progressor” or “Progressor” is a subject that has tested positive for a SARS-CoV virus, such as COVID-19 and develops or is at high risk of developing severe symptoms caused directly or indirectly by the SARS-CoV virus. In some embodiments, a progressor is defined as 1) a subject who was hospitalized for a reason either directly or indirectly (e.g., worsening of a pre-existing condition) related to the SARS-CoV virus or 2) a subject receiving outpatient treatment for a condition either directly or indirectly related to the SARS-CoV virus. In some embodiments, a progressor may include a subject that receives supplemental treatments to treat the SARS-CoV virus and its respective symptoms, not including over-the-counter (OTC) medications. For example, a progressor may receive supplemental oxygen, intravenous fluids, diuretics, anticoagulation, increased doses of medications for pre-existing condition(s), and/or antibiotics.

As used herein, a “non-progressor” or “Non-progressor” is a subject that has tested positive for the SARS-CoV virus and does not develop or is at low risk of developing severe symptoms caused directly or indirectly by the SARS-CoV virus. In some embodiments, non-progressors include subjects that only receive OTC medications for the treatment of the disease. OTC medications may include antipyretics, antitussives, and analgesics. In some embodiments, a non-progressor includes a subject that does not develop advanced symptoms within 28 days of testing positive for the SARS-CoV virus.

As used herein, “SARS-CoV viruses” refer to severe acute respiratory syndrome (SARS)-associated coronavirus. SARS-CoV viruses include any coronavirus that can cause a disease such as SARS. In some embodiments, the SARS-CoV virus is “COVID-19,” refers generally to coronavirus disease of 2019, and includes any variants thereof. For example, COVID-19 includes alpha, delta, epsilon, and any other variants that may arise therefrom. A subject who has tested positive for COVID-19 includes any subject who has received a positive test from any COVID-19 test known to one of skill in the art, including nucleic acid amplification tests, such as polymerase chain reaction (PCR) tests, and antigen tests. The tests may be performed by a technician or by the subject, and may be performed in a clinical setting or an informal setting (e.g., at home).

### Methods for Obtaining a Signature for Identifying a Subject at Risk of a Severe Reaction to a SARS-CoV Virus

The present technology includes a method for determining a signature for identifying a subject at risk of severe reaction to a SARS-CoV virus comprising identifying a subject who has tested positive for a SARS-CoV virus, obtaining a sample from the subject, identifying a cell or tissue from a sample, and performing one or more assays on the cell or tissue.

In some embodiments, identifying a subject who has tested positive for a SARS-CoV virus includes determining whether the subject is or has been infected with a SARS-COV virus. A subject that tests positive for a SARS-CoV virus includes a subject who tests positive using any a SARS-CoV virus test known to one of skill in the art, including nucleic acid amplification tests, such as PCR tests, and antigen tests. The a SARS-CoV virus test may be performed by a technician or by the subject, and may be performed in a clinical setting or an informal setting (i.e. in-home).

In some embodiments, a sample is obtained from the subject. The sample may be a liquid sample, such as a blood, liquid bone marrow, urine, cerebral spinal fluid, mucous, or saliva. The sample may be a solid biopsy, such as lung, muscle, liver, kidney, pancreas, thymus, thyroid, spleen, intestine, bone, or bone marrow. In some embodiments, a blood sample is obtained from the subject. The sample may be obtained by collecting a sample from the subject, for example by blood draw. The sample may also be obtained by receiving the sample from a party who collected the sample from the subject.

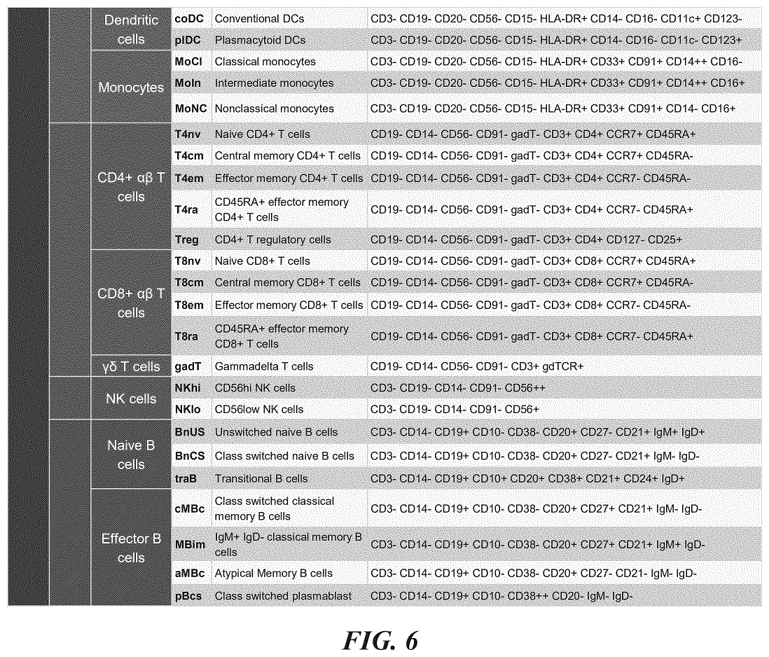

In some embodiments, the sample is processed to isolate or identify a particular tissue or cell type in the sample. As used herein, “identifying” includes classifying or categorizing a tissue or cell. As used herein, “isolating” includes identifying a tissue or cell and separating it from other tissues or cells. A blood sample may be processed to isolate or identify plasma, white blood cells, peripheral blood mononuclear cells (PBMCs), platelets, serum, and/or red blood cells. In some embodiments, a cell is identified or isolated from the blood sample of the subject. In some aspects, the cell is a monocyte subset, such as an intermediate monocyte (MoIn), non-classical monocyte (MoNC), or classical monocyte (MoCl). In some embodiments, the cell is a lymphocyte subset. Tissues may be processed using any technique known to one of skill in the art. A cell may be identified or isolated using any method known to one of ordinary skill in the art, such as flow cytometry or fluorescence-activated cell sorting (FACS). Immune cells may be identified using cell-surface markers known to one of skill in the art. In some embodiments, immune cells are identified using the cell-surface markers listed in FIG. 6.

A classical monocyte may be a cell that has the following cell-surface marker profile: CD3− CD19− CD20− CD56− CD15− HLA-DR+ CD33+ CD91+ CD14++ CD16−. An intermediate monocyte may be a cell that has the following cell-surface marker profile: CD3− CD19− CD20− CD56− CD15− HLA-DR+ CD33+ CD91+ CD14++ CD16+. A nonclassical monocyte may be a cell that has the following cell-surface marker profile: CD3− CD19− CD20− CD56− CD15− HLA-DR+ CD33+ CD91+ CD14− CD16−. A CD8+ effector memory T cell may be a cell that has the following cell-surface marker profile: CD19− CD14− CD56− CD91− gadT− CD3+ CD8+ CCR7− CD45RA−.

In some embodiments, one or more assays may be performed on the cell, tissue, or sample to detect a feature in the subject. The assay may include real time PCR (RT-PCR), quantitative PCR (qPCR), assays for transposase-accessible chromatin with sequencing (ATAC-seq), RNA sequencing (RNA-seq), tagmentation of prime editor sequencing (TaPE-seq), whole genome sequencing (WGS), flow cytometry, fluorescence-activated cell sorting (FACS), droplet digital PCR (ddPCR), histology, immunostaining, magnetic sorting, enzyme-linked immunosorbent assays (ELISAs), Luminex® assays, and other assays known to one of skill in the art.

The feature may be determined from one or more cells, molecules, or compounds from the tissue sample, such as a blood sample. For example, the feature may be a concentration of a protein, peptide, metabolite, enzyme, cytokine, or other molecule or macromolecule detected in the tissue sample. In some embodiments, a feature includes a concentration of cytokines from the serum of a subject. A concentration of a protein, peptide, metabolite, enzyme, cytokine, or other molecule or macromolecule may be determined using a technique known to one of skill in the art.

In some embodiments, the feature may include a genetic or epigenetic characteristic of a cell or population of cells in the sample. The feature may include any data acquired through multi-omic profiling. In some embodiments, differentially expressed genes (DEGs) may be measured in a cell or population of cells in the sample. In some embodiments, differentially accessible genes (DAGs) are measured in the cell or population of cells. In some embodiments, genome-wide chromatin accessibility is measured in the cell or population of cells.

In some embodiments, a feature may be determined from measuring the frequency of certain cell types in the sample. For example, a feature may include a frequency of a certain cell type in a subject's blood sample. In some embodiments, a feature includes a frequency of a monocyte subset, for example, the ratio of a monocyte subset to total monocytes. The feature may be a ratio of MoINs and/or MoNCs to total monocytes. In some embodiments, the feature may be a ratio of MoINs and/or MoNCs to total PBMCs.

In some embodiments, the method for determining a signature for identifying a subject at risk of a severe reaction to a SARS-CoV virus comprises:
(i) obtaining a blood sample from a subject who has tested positive for the SARS-CoV virus;
(ii) identifying a cell from the blood sample of the subject, wherein the cell is an intermediate monocyte (MoIn), non-classical monocyte (MoNC), classical monocyte (MoCl), and/or CD8+ effector memory T cell; and
(iii) performing an assay on the cell to detect one or more features, wherein the signature comprises the one or more features.

In some embodiments, the method for determining a signature for identifying a subject at risk of a severe reaction to SARS-CoV virus comprises:
(i) obtaining a blood sample from a subject who has tested positive for the SARS-CoV virus;
(ii) identifying an immunolabeled cell from the blood sample, wherein the immunolabeled cell comprises a cell from the blood sample and an antibody;
(ii) identifying an intermediate monocyte (MoIn), non-classical monocyte (MoNC), classical monocyte (MoCl), and/or CD8+ effector memory T cell from the immunolabeled cell; and (iii) performing an assay on the cell to detect one or more features, wherein the signature comprises the one or more features.

In some embodiments, the method further comprises determining a ratio of the MoIn and/or MoNC to total monocytes.

In some embodiments, the method further comprising measuring a cytokine from serum from the blood sample.

The signature of the present technology comprises one or more features detected using the method of the present technology. The features may include cellular, molecular, or multi-omic data that reflects the biological status of the subject.

In some embodiments, a feature is an elevated serum concentration of a first cytokine. The elevated serum concentration may be a subject concentration of the first cytokine that is greater by a predetermined third amount than a control concentration of the first cytokine. As used herein, a “cytokine” refers to a protein or gene product. As used herein, a “subject concentration” refers to the concentration of a cytokine in the subject, and a “control concentration” refers to the concentration of a cytokine in a control non-progressor. In some embodiments, the predetermined third amount is a value equal to or greater in magnitude to the log 2FC (log 2 fold change) for each differentially expressed cytokine shown in Table 1.

TABLE 1
differentially expressed cytokines in progressors
with false discovery rates and fold change in expression,
shown as log10 fold change (log10FC)

| protein name | FDR | log10FC |
|---|---|---|
| Fractalkine | 0.073666755 | 0.151145554 |
| G-CSF | 0.098295344 | 0.231650722 |
| IFNα2 | 0.079740861 | 0.175250065 |
| IFNγ | 0.041214952 | 0.450032573 |
| IL-13 | 0.079740861 | 0.299788173 |
| IL-15 | 0.035286107 | 0.181286815 |
| IL-17E/IL-25 | 0.079740861 | 0.228810466 |
| IL-18 | 0.073666755 | 0.20128894 |
| IL-1RA | 0.073666755 | 0.195600591 |
| IL-22 | 0.073666755 | 0.333800471 |
| IL-27 | 0.073666755 | 0.174448683 |
| IL-4 | 0.073666755 | 0.303358786 |
| IL-5 | 0.074431432 | 0.186004845 |
| IL-6 | 0.035286107 | 0.466319549 |
| IL-7 | 0.035286107 | 0.299265326 |
| IP-10 | 0.074431432 | 0.455564596 |
| M-CSF | 0.079740861 | 0.174610111 |
| MCP-3 | 0.091706264 | 0.195418538 |
| MIG | 0.035286107 | 0.241693789 |
| PDGF-AA | 0.035286107 | 0.260721364 |
| PDGF-AB/BB | 0.074431432 | 0.157523674 |
| TGFα | 0.079740861 | 0.269065996 |
| TNFα | 0.049383454 | 0.241942773 |
| VEGF-A | 0.073666755 | 0.267058045 |

In some embodiments, the first cytokine comprises or is selected from a group consisting of Fractalkine, G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα. In some embodiments, the elevated serum concentration comprises a subject concentration of a first cytokine that is greater than a predetermined concentration of the first cytokine. The predetermined concentration of the first cytokine may be 0.2-5, 5-10, 10-100, or 100-1000 pg/mL.

In some embodiments, the first cytokine comprises IL-6. The predetermined IL-6 concentration may be the mean IL-6 concentration of a non-progressor. The predetermined IL-6 concentration may be about 1 pg/mL, 2 pg/mL, 3 pg/mL, 4 pg/mL, 5 pg/mL, 6 pg/mL, 7 pg/mL, 8 pg/mL, 9 pg/mL, or 10 pg/mL.

In some embodiments, the first cytokine comprises IFNγ. The predetermined IFNγ concentration may be the mean IFNγ concentration of a non-progressor. The predetermined IFNγ concentration may be about 1 pg/mL, 2 pg/mL. 3 pg/mL, 4 pg/mL. 5 pg/mL, 6 pg/mL, 7 pg/mL, 8 pg/mL, 9 pg/mL, or 10 pg/mL.

In some embodiments, the first cytokine comprises IL-7. The predetermined IL-7 concentration may be the mean IL-7 concentration of a non-progressor. The predetermined IL-7 concentration may be about 0.2 pg/mL, 0.3 pg/mL, 0.4 pg/mL, 0.5 pg/mL, 0.6 pg/mL, 0.7 pg/mL, 0.8 pg/mL, 0.9 pg/mL, 1 pg/mL, 2 pg/mL, 3 pg/mL, 4 pg/mL, or 5 pg/mL.

In some embodiments, the first cytokine comprises PDGF-AA. The predetermined PDGF-AA concentration may be the mean PDGF-AA concentration of a non-progressor. The predetermined PDGF-AA concentration may be about 100 pg/mL, 200 pg/mL. 300 pg/mL, 400 pg/mL, 500 pg/mL, 600 pg/mL, 700 pg/mL, 800 pg/mL, 900 pg/mL, or 1000 pg/mL.

In some embodiments, the first cytokine comprises TNF. The predetermined TNF concentration may be the mean TNF concentration of a non-progressor. The predetermined TNF concentration may be about 10 pg/mL, 20 pg/mL. 30 pg/mL, 40 pg/mL. 50 pg/mL, 60 pg/mL, 70 pg/mL, 80 pg/mL, 90 pg/mL, or 100 pg/mL.

In some embodiments, the first cytokine comprises IL-15. The predetermined IL-15 concentration may be the mean IL-15 concentration of a non-progressor. The predetermined IL-15 concentration may be about 6 pg/mL, 6.5 pg/mL, 7 pg/mL. 7.5 pg/mL, 8 pg/mL, 8.5 pg/mL, 9 pg/mL, 9.5 pg/mL, or 10 pg/mL.

In some embodiments, a feature may include an elevated serum concentration of one or more first cytokines. The subject may have an elevated serum concentration of one or more first cytokines if the subject has one or more elevated cytokines selected from the group consisting of G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα. The subject may have elevated serum concentrations of one or more first cytokines if the subject has 1, 2, 3, 4, 5, 6, 7, 8, 9, 10, 11, 12, 13, 14, 15, 16, 17, 18, 19, 20, 21, or 22 elevated cytokines selected from the group consisting of G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα.

In some embodiments, a feature comprises an elevated ratio of IL-6 to a second cytokine. The elevated ratio of IL-6 to a second cytokine is a subject ratio of a subject IL-6 concentration to a subject concentration of the second cytokine that is greater than a control ratio of a control IL-6 concentration to a control concentration of the second cytokine. As used herein, a “subject ratio of a subject IL-6 concentration” refers to the ratio of the concentration of IL-6 to the concentration of the second cytokine in the subject, and a “control ratio of a control IL-6 concentration” refers to the ratio of the concentration of IL-6 to the concentration of the second cytokine in a control non-progressor. The second cytokine may comprise or be selected from the group consisting of G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα.

In some embodiments, the second cytokine comprises or is selected from the group consisting of IFNα2, IFNγ, TNF, IL-17A, and IL-10. In some embodiments, the elevated ratio comprises an IL-6:IFNα2 ratio greater than a predetermined IL-6:IFNα2 ratio. The elevated ratio may comprise an IL-6:IFNγ ratio greater than a predetermined IL-6:IFNγ. The elevated ratio may comprise an IL-6:TNF ratio greater than a predetermined IL-6:TNF. The elevated ratio may comprise an IL-6:IL-17A ratio greater than a predetermined IL-6:IL-17A ratio. The elevated ratio may comprise an IL-6:IL-10 ratio that is greater than a predetermined IL-6:IL-10 ratio.

The predetermined IL-6:IFNα2 ratio may be the mean IL-6:IFNα2 ratio of a non-progressor. The predetermined IL-6:IFNα2 ratio may be about 6 pg/mL, 7 pg/mL, 8 pg/mL, 9 pg/mL, 10 pg/mL, 11 pg/mL, 12 pg/mL, 13 pg/mL, 14 pg/mL, 15 pg/mL, 16 pg/mL, 17 pg/mL, 18 pg/mL, 19 pg/mL, or 20 pg/mL.

The predetermined IL-6:IFNγ ratio may be the mean IL-6:IFNγ ratio of a non-progressor. The predetermined IL-6:IFNγ ratio may be about 10 pg/mL, 15 pg/mL, 20 pg/mL, 25 pg/mL, 30 pg/mL, 35 pg/mL, or 40 pg/mL.

The predetermined IL-6:TNF ratio may be the mean IL-6:TNF ratio of a non-progressor. The predetermined IL-6:TNF ratio may be about 6 pg/mL. 7 pg/mL. 8 pg/mL, 9 pg/mL, 10 pg/mL, 11 pg/mL, 12 pg/mL. 13 pg/mL, 14 pg/mL. 15 pg/mL, 16 pg/mL, 17 pg/mL, 18 pg/mL, 19 pg/mL, or 20 pg/mL.

The predetermined IL-6:IL-17A ratio may be the mean IL-6:IL-17A ratio of a non-progressor. The predetermined IL-6:IL-17A ratio may be about 10 pg/mL, 15 pg/mL, 20 pg/mL. 25 pg/mL, 30 pg/mL. 35 pg/mL, or 40 pg/mL.

The predetermined IL-6:IL-10 ratio may be the mean IL-6:IL-10 ratio of a non-progressor. The predetermined IL-6:IL-10 ratio may be about 10 pg/mL, 15 pg/mL, 20 pg/mL. 25 pg/mL, 30 pg/mL. 35 pg/mL, or 40 pg/mL.

In some embodiments, a feature may include an elevated ratio of IL-6 to one or more second cytokines. The subject may have an elevated ratio of IL-6 to one or more second cytokines if the subject has one or more elevated ratios of IL-6 to a cytokine selected from the group consisting of IFNα2, IFNγ, TNF, IL-17A, and IL-10. The subject may have an elevated ratio of IL-6 to one or more second cytokines if the subject has 1, 2, 3, 4, or 5 elevated ratios of IL-6 to a cytokine selected from the group consisting of IFNα2, IFNγ, TNF, IL-17A, and IL-10.

In some embodiments, a feature may include an elevated ratio of IL-6 to one or more second cytokines. The subject may have an elevated ratio of IL-6 to one or more second cytokines if the subject has one or more elevated ratios of IL-6 to a cytokine selected from the group consisting of G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα. The subject may have an elevated ratio of IL-6 to one or more second cytokines if the subject has 1, 2, 3, 4, 5, 6, 7, 8, 9, 10, 11, 12, 13, 14, 15, 16, 17, 18, 19, 20, 21, or 22 elevated ratios of IL-6 to a cytokine selected from the group consisting of G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα.

In some embodiments, a feature comprises a reduced ratio of an intermediate monocyte subclass (MoIn) and/or non-classical monocyte subclass (MoNC) to total monocytes. In some embodiments, the reduced ratio of the MoIn and/or MoNC to total monocytes is a subject ratio of MoIn to total monocytes that is lower than a control ratio of MoIn to total monocytes and/or a subject ratio of MoNC to total monocytes that is lower than a control ratio MoNC to total monocytes. As used herein, a “subject ratio of the MoIn and/or MoNC” refers to the ratio of the number of MoIn and/or MoNC in a subject to total monocytes in the subject, and a “control ratio of the MoIn and/or MoNC” refers to the ratio of the number of MoIn and/or MoNC to total monocytes in a control non-progressor. In some embodiments, the reduced ratio of the MoIn and/or MoNC to total monocytes is a subject ratio of MoIn and/or MoNC monocytes that is lower by a predetermined sixth amount than the ratio of the MoIn and/or MoNC to total monocytes in a control subject. The predetermined sixth amount may be a value that is equal to or greater in magnitude to the log 2FC (log 2 fold change) for each differentially expressed cytokine shown in Table 2.

TABLE 2
Frequency of monocyte subtypes in progressors with
false discovery rates and fold change in expression,
shown as log10 fold change (log10FC)

| monocyte_freq | FDR | log10FC |
|---|---|---|
| (MoNC + MoIn)/(MoCl + MoIn + | 0.054923231 | −0.603025887 |
| MoNC) | | |
| MoCl/(MoCl + MoIn + MoNC) | 0.368843173 | 0.058343191 |
| MoIn/(MoCl + MoIn + MoNC) | 0.058595642 | −0.563526119 |
| MoNC/(MoCl + MoIn + MoNC) | 0.338266732 | −1.051856348 |

In some embodiments, the ratio of MoINs to total monocytes is a subject ratio of MoIn to total monocytes that is lower than a predetermined MoIn ratio. The ratio of the MoNCs to total monocytes may be a ratio of MoNC to total monocytes that is lower than a predetermined MoNC ratio.

The predetermined MoIn ratio may be the mean MoIn ratio of a non-progressor. The predetermined MoIn ratio may be measured as the percent of total monocytes that are MoIn cells. In some embodiments, the predetermined MoIn ratio may be about 10%, 15%, 20%, 25%, 30%, 35% 40%, 45%, 50%, 55%, 60%, 65%, 70%, 75%, 80%, 85% or 90% of the total monocytes.

The predetermined MoNC ratio may be the mean MoNC ratio of a non-progressor. The predetermined MoNC ratio may be measured as the percent of total monocytes that are MoNC cells. In some embodiments, the predetermined MoNC ratio may be about 0.5%, 0.6%, 0.7%, 0.8%, 0.9%, 1% 5%, 10%, 15%, 20%, 25%, 30%, 35%, 40%, 45%, or 50% of the total monocytes.

In some embodiments, a feature comprises a ratio of MoIn and MoNC cells to total monocytes. The ratio of MoIn and MoNC cells to total monocytes may be a subject ratio of MoIn and MoNC to total monocytes that is lower than a predetermined ratio of MoIn and MoNC cells. The predetermined ratio of MoIn and MoNC cells may be the mean ratio of MoIn and MoNC cells of a non-progressor. The predetermined ratio of MoIn and MoNC cells may be measured as the percent of total monocytes that are MoIn or MoNC cells. In some embodiments, the predetermined ratio of MoIn and MoNC cells may be about 10%, 15%, 20%, 25%, 30%, 35% 40%, 45%, 50%, 55%, 60%, 65%, 70%, 75%, 80%, 85% or 90% of the total monocytes.

In some embodiments, a feature comprises a first DEG in a monocyte subclass. The monocyte may be MoIn, MoNC, and/or a classical monocyte (MoCl). The DEG may be detected using a whole genome approach, such as RNA sequencing. Alternatively, the DEG may be detected using a targeted approach, such as RT-PCR.

In some embodiments, the first DEG is a gene that is expressed in a subject at a level that is significantly different from the level that it is expressed in a control non-progressor. The first DEG may be expressed at a level that is different by a predetermined first amount than the expression of the first DEG in a control subject (non-progressor). The first DEG may be expressed at a level that is at least about 1.5, 1.75, 2, 2.25, 2.5, 2.75, or 3-fold greater or less than the expression of the first DEG in a control non-progressor. In some embodiments, the predetermined first amount is a value equal to or greater in magnitude to the log 2FC (log 2 fold change) for each DEG shown in Table 3. As used herein, “greater in magnitude” to the log 2FC refers to a value that is further from zero from the log 2FC value.

TABLE 3
DEGs for monocyte subsets with false discovery rates and fold
change in expression, shown as log2 fold change (log2FC)

| gene_name | subset | FDR | log2FC |
|---|---|---|---|
| ASGR2 | MoIn | 0.044085203 | 1.177573749 |
| C1QA | MoIn | 0.084243144 | 1.116680639 |
| C1QB | MoIn | 0.084243144 | 1.400721647 |
| CD163 | MoIn | 0.018674284 | 1.521665038 |
| CLU | MoIn | 0.000389977 | 2.513639873 |
| COX1 | MoIn | 0.0341184852 | −0.819526481 |
| GPX1 | MoIn | 0.044085203 | 0.657548516 |
| HLX | MoIn | 0.059497043 | 1.131141546 |
| HMGB2 | MoCI | 0.007452181 | 0.973990235 |
| IL1R2 | MoCI | 0.016557931 | 2.054968513 |
| LAIR2 | MoNC | 0.044682282 | 2.969441169 |
| LYN | MoIn | 0.044085203 | −0.477891623 |
| MCEMP1 | MoIn | 0.084243144 | 1.34465237 |
| MT-CO1 | MoIn | 0.034118452 | −0.819526481 |
| RAB13 | MoIn | 0.043233735 | 1.204335178 |
| RNASE1 | MoIn | 0.000379009 | 2.316144948 |
| SAP30 | MoCl | 0.007452181 | 1.615755742 |
| S100A12 | MoIn | 0.007169683 | 1.685685896 |
| S100A9 | MoIn | 0.095485451 | 0.904226624 |
| SVIL | MoIn | 0.043233735 | −0.76726872 |
| THBD | MoIn | 0.084243144 | 1.253768519 |
| TRPM2 | MoIn | 0.084243144 | 1.007382949 |
| VSIG4 | MoIn | 0.044085203 | 2.172744993 |

In some embodiments, the first DEG comprises a gene associated with coagulation, the complement cascade, and/or pathogen phagocytosis.

In some embodiments, the monocyte subclass is MoCl and the first DEG comprises or consists of a gene in the IL1R2, SAP30, and/or HMGB2 pathway. The first DEG may comprise or consist of IL1R2, SAP30, and/or HMGB2 in the MoCl.

In some embodiments, a feature may include one or more first DEGs in a MoCl. The one or more first DEGs may be one or more differentially expressed genes, where the genes are selected from the group consisting of IL1R2, SAP30, and/or HMGB2. The one or more first DEGs may be 1, 2, or 3 differentially expressed genes, where the genes are selected from the group consisting of IL1R2, SAP30, and HMGB2.

In some embodiments, the monocyte subclass is MoIn and the first DEG comprises or consists of a gene in the ASGR2, C1QA, C1QB, CD163, CLU, COX1, GPX1, HLX, LYN, MCEMP1, MT-CO1, RAB13, RNASE1, S100A12, S100A9, SVIL, THBD, TRPM2, and/or VSIG4 pathway. The first DEG may comprise or consist of ASGR2, C1QA, C1QB, CD163, CLU, COX1, GPX1, HLX, LYN, MCEMP1, MT-CO1, RAB13, RNASE1, S100A12, S100A9, SVIL, THBD, TRPM2, and/or VSIG4 in the MoIn. In some preferred embodiments, the first DEG may comprise or consist of C1QB, MCEMP1, S100A9, THBD, and/or TRPM. In other preferred embodiments, the first DEG may comprise or consist of COX1, SVIL, LYN, GPX1, ASGR2, RAB13, CD163, S100A12, VSIG4, RNASE1, and/or CLU.

In some embodiments, a feature may include one or more first DEGs in a MoIn. The one or more first DEGs may be one or more differentially expressed genes, where the genes are selected from the group consisting of ASGR2, C1QA, C1QB, CD163, CLU, COX1, GPX1, HLX, LYN, MCEMP1, MT-CO1, RAB13, RNASE1, S100A12, S100A9, SVIL, THBD, TRPM2, and/or VSIG4. The one or more first DEGs may be 1, 2, 3, 4, 5, 6, 7, 8, 9, 10, 11, 12, 13, 14, 15, 16, 17, or 18 differentially expressed genes, where the genes are selected from the group consisting of ASGR2, CIQA, C1QB, CD163, CLU, COX1, GPX1, HLX, LYN, MCEMP1, MT-CO1, RAB13, RNASE1, S100A12, S100A9, SVIL, THBD, TRPM2, and/or VSIG4. In some preferred embodiments, the one or more first DEGs may be 1, 2, 3, 4, or 5 differentially expressed genes, where the genes are selected from the group consisting of C1QB, MCEMP1, S100A9, THBD, and/or TRPM. In other preferred embodiments, the one or more first DEGs may be 1, 2, 3, 4, 5, 6, 7, 8, 9, 10, or 11 differentially expressed genes, where the genes are selected from the group consisting of COX1, SVIL, LYN, GPX1, ASGR2, RAB13, CD163, S100A12, VSIG4, RNASE1, and/or CLU.

In some embodiments, the monocyte subclass is MoNC and the first DEG comprises or consists of a gene in the LAIR2 pathway. The first DEG may comprise or consist of LAIR2 in the MoNC.

In some embodiments, the first DEG may be located a predetermined first distance from a differentially accessible region (DAR). The DAR may be detected using a whole genome approach, such as ATAC-seq. Alternatively, the DEG may be detected using a targeted approach, such as RNA-seq.

The DAR may be a region of the genome in a subject that has a chromatin accessibility that is significantly different from the chromatin accessibility of the region of the genome in a control non-progressor. The DAR may be a region of the genome in a subject that has a chromatin accessibility that is different than the mean chromatin accessibility for the region in control non-progressors. The DAR may be a region of the genome in a subject that has a chromatin accessibility that is different from the region of the genome in a control non-progressor with a false discovery rate (FDR) less than 0.5 or 0.1. In some embodiments, the DAR is a region of the genome in a subject that has a chromatin accessibility that is different by a predetermined fifth amount than the chromatin accessibility in the region of the genome of a control subject. In some embodiments, the predetermined fifth amount is a value equal to or greater in magnitude to the log 2FC (log 2 fold change) for each DEG shown in Table 4.

TABLE 4
DARs in progressors with false discovery rates and fold change
in chromatin accessibility, shown as log2 fold change (log2FC)

| peak_id | subset_code | FDR | log2FC |
|---|---|---|---|
| chr10: 127797544 | MoIn | 0.004109019 | 1.380307905 |
| chr10: 132364433 | MoIn | 0.047689688 | 1.119758215 |
| chr10: 99403877 | MoIn | 0.038889049 | 1.026364112 |
| chr11: 1016892 | MoIn | 0.001329214 | 1.091702125 |
| chr14: 63864506 | MoIn | 0.029901029 | 1.080089078 |
| chr16: 57000725 | MoIn | 0.040591245 | 1.031574784 |
| chr16: 57277794 | MoIn | 0.003895969 | 1.078096293 |
| chr19: 37293653 | MoIn | 0.008366099 | 1.567204385 |
| chr1: 206803446 | MoIn | 0.041600755 | 1.034884446 |
| chr1: 2652330 | MoIn | 0.001084074 | 1.848202883 |
| chr1: 2654120 | MoIn | 0.001639563 | 1.65199286 |
| chr20: 31106819 | MoIn | 0.005424789 | 1.328418728 |
| chr20: 31157809 | MoIn | 0.03414009 | 1.269894075 |
| chr20: 31185086 | MoIn | 0.039673914 | 1.343827353 |
| chr20: 31186558 | MoIn | 0.021742413 | 1.262888024 |
| chr20: 31189267 | MoIn | 0.021960009 | 1.292266285 |
| chr20: 31241753 | MoIn | 0.003021477 | 1.490564678 |
| chr21: 7956259 | MoIn | 0.002182594 | 1.455990237 |
| chr22: 11028245 | MoIn | 0.010532912 | 1.095315118 |
| chr22: 11056283 | MoIn | 0.024284484 | 1.041334691 |
| chr3: 128243047 | MoIn | 0.002770254 | 1.153671879 |
| chr4: 3815017 | MoIn | 0.002509135 | 1.449124215 |
| chr5: 179344116 | MoIn | 0.013667085 | 1.069036639 |
| chr6: 157312993 | MoIn | 0.0002007 | 1.107144807 |
| chr7: 100958167 | MoIn | 0.014934557 | 1.030765375 |
| chr7: 100958631 | MoIn | 0.020308214 | 1.012836137 |
| chr7: 139682631 | MoIn | 0.003827325 | 1.035762098 |
| chr9: 90821442 | MoIn | 0.029163097 | −1.024659658 |

In some embodiments, the predetermined first distance is less than 300,000 250,000; 200,000; 150,000; 100,000; 75,000; 50,000; or 25,000 nucleotides.

In some embodiments, a feature comprises a second DEG in a CD8+ effector memory T cell. The second DEG may be detected using a whole genome approach, such as RNA sequencing. Alternatively, the second DEG may be detected using a targeted approach, such as RT-PCR.

In some embodiments, the second DEG is a gene that is expressed in a subject at a level that is significantly different from the level that the second DEG is expressed in a control non-progressor. The second DEG may be expressed at a level that is different by a predetermined second amount than the expression of the second DEG in a control subject (non-progressor). The second DEG may be expressed at a level that is at least about 1.5, 1.75, 2, 2.25, 2.5, 2.75, or 3-fold greater or less than the expression of the second DEG in a control non-progressor. In some embodiments, the predetermined second amount is a value equal to or greater in magnitude to the log 2FC (log 2 fold change) for each DEG shown in Table 5.

TABLE 5
DEGs for CD8 effector memory T cells (T8em) with
false discovery rates and fold change in expression,
shown as log2 fold change (log2FC)

| gene_name | subset | FDR | log2FC |
|---|---|---|---|
| ACAA2 | T8em | 0.04667508 | 0.506586498 |
| ACAT1 | T8em | 0.096549893 | 0.468558097 |
| ACAT2 | T8em | 0.047752767 | 0.499732537 |
| ACTB | T8em | 0.074239502 | 0.469518994 |
| ADA | T8em | 0.079454835 | 0.401647014 |
| AFMID | T8em | 0.087619957 | 0.803797433 |
| AHCY | T8em | 0.050391594 | 0.623634337 |
| AHSA1 | T8em | 0.047752767 | 0.42969286 |
| ALOX5 | T8em | 0.079454835 | −1.460986534 |
| ANKRD23 | T8em | 0.066494856 | −0.705341663 |
| ANXA2 | T8em | 0.079454835 | 0.502256256 |
| ANXA5 | T8em | 0.068138484 | 0.514307772 |
| APOBEC3B | T8em | 0.055479352 | 1.810247966 |
| ARPC1A | T8em | 0.063834362 | 0.273825861 |
| ASNA1 | T8em | 0.05204853 | 0.400845883 |
| ATP5C1 | T8em | 0.054694839 | 0.368781164 |
| ATP5E | T8em | 0.079454835 | 0.392298256 |
| ATP6V1F | T8em | 0.055479352 | 0.285787192 |
| BIRC5 | T8em | 0.05225934 | 1.30856282 |
| BOP1 | T8em | 0.068138484 | 0.6610501 |
| C19orf54 | T8em | 0.068138484 | 0.744949002 |
| CALM3 | T8em | 0.068138484 | 0.348178676 |
| CCDC130 | T8em | 0.070241723 | −0.317131898 |
| CCL3 | T8em | 0.07596362 | 1.093356105 |
| CCT4 | T8em | 0.093749892 | 0.307422021 |
| CCT8 | T8em | 0.096016638 | 0.283739022 |
| CD53 | T8em | 0.08497567 | 0.503995625 |
| CD59 | T8em | 0.074553932 | 0.54335109 |
| CD70 | T8em | 0.08497567 | 0.756220651 |
| CDK4 | T8em | 0.087619957 | 0.638324924 |
| CDKN3 | T8em | 0.08050247 | 1.125424672 |
| CENPM | T8em | 0.085136265 | 0.887282966 |
| CHEK1 | T8em | 0.047752767 | 0.736972757 |
| CHI3L2 | T8em | 0.0456385 | 1.245947471 |
| CIRBP | T8em | 0.07095605 | −0.3075265 |
| CISD1 | T8em | 0.072644147 | 0.623563039 |
| CKS1B | T8em | 0.079728977 | 0.727037845 |
| CLASP1 | T8em | 0.07095605 | −0.397762522 |
| CLIC1 | T8em | 0.06892096 | 0.500029638 |
| CLTA | T8em | 0.085847032 | 0.420675516 |
| CNIH1 | T8em | 0.06892096 | 0.467383068 |
| COL6A2 | T8em | 0.04667508 | −1.194086318 |
| COPS5 | T8em | 0.074553932 | 0.281026751 |
| COX5A | T8em | 0.079454835 | 0.382847861 |
| COX6A1 | T8em | 0.08337244 | 0.312669065 |
| CTSC | T8em | 0.074482152 | 0.541688077 |
| DBI | T8em | 0.087296357 | 0.339443707 |
| DOCK10 | T8em | 0.088912225 | −0.301400133 |
| EIF1AD | T8em | 0.06892096 | −0.484772076 |
| ENO1 | T8em | 0.081935779 | 0.449605687 |
| ENTPD4 | T8em | 0.06892096 | −0.378610926 |
| ERAL1 | T8em | 0.05204853 | 0.456423041 |
| FAM160B2 | T8em | 0.08497567 | −0.35446184 |
| FARP2 | T8em | 0.081911285 | −0.52851908 |
| FDXR | T8em | 0.048906746 | 0.925954166 |
| FKBP1A | T8em | 0.08497567 | 0.482602894 |
| GALK1 | T8em | 0.079454835 | 0.586851565 |
| GAPDH | T8em | 0.08227374 | 0.357300295 |
| GGT7 | T8em | 0.05225934 | −0.867874757 |
| GIGYF1 | T8em | 0.08227374 | −0.308954511 |
| GLRX3 | T8em | 0.047752767 | 0.381167475 |
| GNG5 | T8em | 0.068138484 | 0.39813696 |
| GTF2A2 | T8em | 0.048906746 | 0.35904146 |
| GTF2IRD2 | T8em | 0.04667508 | −0.606499272 |
| GTF3C6 | T8em | 0.054694839 | 0.408722564 |
| GZMA | T8em | 0.079728977 | 0.659834306 |
| HADH | T8em | 0.06892096 | 0.658762847 |
| HAUS1 | T8em | 0.08497567 | 0.574253556 |
| HDHD3 | T8em | 0.090256298 | −0.56915369 |
| HIST1H2BD | T8em | 0.06892096 | 0.843449432 |
| HMOX1 | T8em | 0.054033083 | 1.416700239 |
| HN1 | T8em | 0.043055887 | 0.641017021 |
| HNRNPC | T8em | 0.068138484 | 0.25540634 |
| HPRT1 | T8em | 0.055479352 | 0.558685265 |
| IFI27 | T8em | 0.079728977 | 1.504354202 |
| ISOC2 | T8em | 0.05204853 | 0.724468564 |
| LAMTOR1 | T8em | 0.052870596 | 0.26902008 |
| LMNB2 | T8em | 0.079454835 | 0.533912233 |
| LYPLA1 | T8em | 0.08337244 | 0.36610366 |
| MBTPS1 | T8em | 0.081954212 | −0.279947448 |
| MCM4 | T8em | 0.063834362 | 0.909219897 |
| MDH1 | T8em | 0.079454835 | 0.372425203 |
| MECP2 | T8em | 0.068339275 | −0.354752612 |
| METTL16 | T8em | 0.079454835 | −0.379113507 |
| MPP7 | T8em | 0.047752767 | −0.830375595 |
| MPST | T8em | 0.088641477 | 0.626879291 |
| MRPL27 | T8em | 0.04667508 | 0.555784604 |
| MRPL42 | T8em | 0.054694839 | 0.457378497 |
| MRPS14 | T8em | 0.090206177 | 0.651816283 |
| MRPS26 | T8em | 0.079454835 | 0.475715407 |
| MTCH2 | T8em | 0.08050247 | 0.420894897 |
| MYL6 | T8em | 0.096016638 | 0.272936058 |
| MYO15B | T8em | 0.088069271 | −0.842835069 |
| NAA38 | T8em | 0.094731459 | 0.412277897 |
| NAT1 | T8em | 0.070241723 | 0.914901561 |
| NDUFA2 | T8em | 0.079454835 | 0.277276778 |
| NDUFB3 | T8em | 0.096016638 | 0.617632051 |
| NDUFB6 | T8em | 0.070241723 | 0.458030557 |
| NFIA | T8em | 0.043055887 | −0.820135197 |
| NHP2 | T8em | 0.086866827 | 0.299852257 |
| NOP10 | T8em | 0.079454835 | 0.393824383 |
| NR2C2 | T8em | 0.070241723 | −0.381327294 |
| NUF2 | T8em | 0.079454835 | 1.104419509 |
| OSTC | T8em | 0.085847032 | 0.384635266 |
| OTOF | T8em | 0.079454835 | 1.487890897 |
| PARK7 | T8em | 0.04667508 | 0.416119313 |
| PGAM1 | T8em | 0.043055887 | 0.430024978 |
| PGAP3 | T8em | 0.047752767 | −0.533291805 |
| PLK1 | T8em | 0.074553932 | 1.367026791 |
| POP4 | T8em | 0.096016638 | 0.293021068 |
| POU6F1 | T8em | 0.047752767 | −0.929346519 |
| PPIA | T8em | 0.088069271 | 0.357427652 |
| PPIL1 | T8em | 0.079454835 | 0.728586466 |
| PRDX1 | T8em | 0.068138484 | 0.509644088 |
| PRDX3 | T8em | 0.068138484 | 0.813261214 |
| PRELID1 | T8em | 0.088285634 | 0.322052088 |
| PSMA2 | T8em | 0.068138484 | 0.404104539 |
| PSMA6 | T8em | 0.055479352 | 0.353594462 |
| PSMB2 | T8em | 0.06892096 | 0.390091628 |
| RHOA | T8em | 0.06892096 | 0.243388315 |
| RNPC3 | T8em | 0.084376783 | −0.299585125 |
| RP11-500M8.7 | T8em | 0.079454835 | 2.015460091 |
| RPA3 | T8em | 0.047752767 | 0.646391447 |
| RPL26L1 | T8em | 0.063217386 | 0.519823668 |
| SDF2 | T8em | 0.096016638 | 0.317909736 |
| SDF2L1 | T8em | 0.087619957 | 0.342225606 |
| SEC11A | T8em | 0.090256298 | 0.296821141 |
| SENP6 | T8em | 0.06892096 | −0.288144102 |
| SESTD1 | T8em | 0.047752767 | 1.058591505 |
| SHFM1 | T8em | 0.070241723 | 0.390241633 |
| SKA2 | T8em | 0.070241723 | 0.589910753 |
| SLC25A5 | T8em | 0.079454835 | 0.445033018 |
| SLC29A2 | T8em | 0.09075494 | −1.151770551 |
| SLC38A6 | T8em | 0.074239502 | 0.665740281 |
| SLK | T8em | 0.079728977 | −0.415958876 |
| SMS | T8em | 0.068138484 | 0.457187714 |
| SNRPB | T8em | 0.070241723 | 0.286060604 |
| SNRPD3 | T8em | 0.086828267 | 0.307061791 |
| SORL1 | T8em | 0.068138484 | −0.355398662 |
| SRSF8 | T8em | 0.079454835 | −0.325350482 |
| STMN1 | T8em | 0.079454835 | 0.90352243 |
| STOML2 | T8em | 0.096016638 | 0.351020613 |
| TAF1 | T8em | 0.08497567 | −0.363967737 |
| TALDO1 | T8em | 0.06892096 | 0.662327341 |
| TIMM13 | T8em | 0.079454835 | 0.521597494 |
| TKT | T8em | 0.079454835 | 0.404732321 |
| TMEM155 | T8em | 0.085281061 | 1.387377243 |
| TNRC6B | T8em | 0.079454835 | −0.304390767 |
| TPGS2 | T8em | 0.088069271 | 0.400094209 |
| TPI1 | T8em | 0.082222875 | 0.415817142 |
| TPX2 | T8em | 0.043055887 | 1.44790992 |
| TSC1 | T8em | 0.094731459 | −0.37633302 |
| TTLL3 | T8em | 0.08497567 | −0.337283263 |
| TUBGCP6 | T8em | 0.079454835 | −0.317688523 |
| TXN | T8em | 0.096016638 | 0.352608399 |
| TXNDC17 | T8em | 0.054033083 | 0.647045957 |
| UBE2L3 | T8em | 0.04667508 | 0.365149248 |
| UCHL3 | T8em | 0.074482152 | 0.488864527 |
| UQCR10 | T8em | 0.068138484 | 0.345105528 |
| UQCRH | T8em | 0.088069271 | 0.307233779 |
| VRK2 | T8em | 0.08497567 | 0.388629562 |
| VTA1 | T8em | 0.090434845 | 0.356414122 |
| ZBTB20 | T8em | 0.079454835 | −0.434248613 |
| ZNF275 | T8em | 0.074553932 | −0.524317539 |
| ZNF395 | T8em | 0.063410766 | −0.519049092 |
| ZNF518A | T8em | 0.096016638 | −0.593149605 |

In some embodiments, the second DEG comprises a gene associated with T cell activation.

In some embodiments, the second DEG comprises or consists of a gene in the TPX2, CHI3L2, SESTD1, FDXR, CHEK1, RPA3, HN1, MRPL27, ACAA2, ACAT2, PGAM1, AHSA1, PARK7, GLRX3, UBE2L3, GTF2A2, PGAP3, GTF2IRD2, NFIA, MPP7, POU6F1, and/or COL6A2 pathway. The second DEG may comprise or consist of TPX2, CHI3L2, SESTD1, FDXR, CHEK1, RPA3, HN1, MRPL27, ACAA2, ACAT2, PGAM1, AHSA1, PARK7, GLRX3, UBE2L3, GTF2A2, PGAP3, GTF2IRD2, NFIA, MPP7, POU6F1, and/or COL6A2.

In some embodiments, a feature may include one or more second DEGs in a CD8+ effector memory T cell. The one or more second DEGs may be one or more differentially expressed genes, where the genes are selected from the group consisting of TPX2, CHI3L2, SESTD1, FDXR, CHEK1, RPA3, HN1, MRPL27, ACAA2, ACAT2, PGAM1, AHSA1, PARK7, GLRX3, UBE2L3, GTF2A2, PGAP3, GTF2IRD2, NFIA, MPP7, POU6F1, and COL6A2. The one or more second DEGs may be 1, 2, 3, 4, 5, 6, 7, 8, 9, 10, 11, 12, 13, 14, 15, 16, 17, 18, 19, 20, 21, or 22 differentially expressed genes, where the genes are selected from the group consisting of TPX2, CHI3L2, SESTD1, FDXR, CHEK1, RPA3, HN1, MRPL27, ACAA2, ACAT2, PGAM1, AHSA1, PARK7, GLRX3, UBE2L3, GTF2A2, PGAP3, GTF2IRD2, NFIA, MPP7, POU6F1, and/or COL6A2.

In some embodiments, a feature may include one or more second DEGs in a CD8+ effector memory T cell. The one or more second DEGs may be one or more differentially expressed genes where the genes are selected from the group consisting of ACAA2, ACAT1, ACAT2, ACTB, ADA, AFMID, AHCY, AHSA1, ALOX5, ANKRD23, ANXA2, ANXA5, APOBEC3B, ARPC1A, ASNA1, ATP5C1, ATP5E, ATP6V1F, BIRC5, BOP1, C19orf54, CALM3, CCDC130, CCL3, CCT4, CCT8, CD53, CD59, CD70, CDK4, CDKN3, CENPM, CHEK1, CHI3L2, CIRBP, CISD1, CKS1B, CLASP1, CLIC1, CLTA, CNIH1, COL6A2, COPS5, COX5A, COX6A1, CTSC, DBI, DOCK10, EIF1AD, ENO1, ENTPD4, ERAL1, FAM160B2, FARP2, FDXR, FKBP1A, GALK1, GAPDH, GGT7, GIGYF1, GLRX3, GNG5, GTF2A2, GTF2IRD2, GTF3C6, GZMA, HADH, HAUS1, HDHD3, HIST1H2BD, HMOX1, HN1, HNRNPC, HPRT1, IFI27, ISOC2, LAMTOR1, LMNB2, LYPLA1, MBTPS1, MCM4, MDH1, MECP2, METTL16, MPP7, MPST, MRPL27, MRPL42, MRPS14, MRPS26, MTCH2, MYL6, MYO15B, NAA38, NAT1, NDUFA2, NDUFB3, NDUFB6, NFIA, NHP2, NOP10, NR2C2, NUF2, OSTC, OTOF, PARK7, PGAM1, PGAP3, PLK1, POP4, POU6F1, PPIA, PPIL1, PRDX1, PRDX3, PRELID1, PSMA2, PSMA6, PSMB2, RHOA, RNPC3, RP11-500M8.7, RPA3, RPL26L1, SDF2, SDF2L1, SEC11A, SENP6, SESTD1, SHFM1, SKA2, SLC25A5, SLC29A2, SLC38A6, SLK, SMS, SNRPB, SNRPD3, SORL1, SRSF8, STMN1, STOML2, TAF1, TALDO1, TIMM13, TKT, TMEM155, TNRC6B, TPGS2, TPI1, TPX2, TSC1, TTLL3, TUBGCP6, TXN, TXNDC17, UBE2L3, UCHL3, UQCR10, UQCRH, VRK2, VTA1, ZBTB20, ZNF275, ZNF395, and ZNF518A. The one or more second DEGs may be 1-10, 10-20, 20-30, 30-40, 40-50, 50-60, 60-70, 80-90, 90-100, 100-110, 110-120, 120-130, 130-140, 140-150, 150-160, 160-166, 1-50, 50-100, or 100-166 differentially expressed genes, where the genes are selected from the group consisting of ACAA2, ACAT1, ACAT2, ACTB, ADA, AFMID, AHCY, AHSA1, ALOX5, ANKRD23, ANXA2, ANXA5, APOBEC3B, ARPC1A, ASNA1, ATP5C1, ATP5E, ATP6V1F, BIRC5, BOP1, C19orf54, CALM3, CCDC130, CCL3, CCT4, CCT8, CD53, CD59, CD70, CDK4, CDKN3, CENPM, CHEK1, CHI3L2, CIRBP, CISD1, CKS1B, CLASP1, CLIC1, CLTA, CNIH1, COL6A2, COPS5, COX5A, COX6A1, CTSC, DBI, DOCK10, EIF1AD, ENO1, ENTPD4, ERAL1, FAM160B2, FARP2, FDXR, FKBP1A, GALK1, GAPDH, GGT7, GIGYF1, GLRX3, GNG5, GTF2A2, GTF2IRD2, GTF3C6, GZMA, HADH, HAUS1, HDHD3, HIST1H2BD, HMOX1, HN1, HNRNPC, HPRT1, IFI27, ISOC2, LAMTOR1, LMNB2, LYPLA1, MBTPS1, MCM4, MDH1, MECP2, METTL16, MPP7, MPST, MRPL27, MRPL42, MRPS14, MRPS26, MTCH2, MYL6, MYO15B, NAA38, NAT1, NDUFA2, NDUFB3, NDUFB6, NFIA, NHP2, NOP10, NR2C2, NUF2, OSTC, OTOF, PARK7, PGAM1, PGAP3, PLK1, POP4, POU6F1, PPIA, PPIL1, PRDX1, PRDX3, PRELID1, PSMA2, PSMA6, PSMB2, RHOA, RNPC3, RP11-500M8.7, RPA3, RPL26L1, SDF2, SDF2L1, SEC11A, SENP6, SESTD1, SHFM1, SKA2, SLC25A5, SLC29A2, SLC38A6, SLK, SMS, SNRPB, SNRPD3, SORL1, SRSF8, STMN1, STOML2, TAF1, TALDO1, TIMM13, TKT, TMEM155, TNRC6B, TPGS2, TPI1, TPX2, TSC1, TTLL3, TUBGCP6, TXN, TXNDC17, UBE2L3, UCHL3, UQCR10, UQCRH, VRK2, VTA1, ZBTB20, ZNF275, ZNF395, and ZNF518A.

In a preferred embodiment, a feature may include one or more second DEGs in a CD8+ effector memory T cell and the one or more second DEGs may be one or more differentially expressed genes where the genes are selected from the group consisting of ACAA2, BIRC5, CD53, CD59, CISD1, COL6A2, FDXR, GTF2IRD2, HADH, HDHD3, IFI27, ISOC2, MRPL27, MYO15B, NFIA, OTOF, PRDX1, RPL26L1, SESTD1, TIMM13, and TXNDC17. The one or more second DEGs may be 1-5, 6-10, 11-15, or 16-21 differentially expressed genes, where the genes are selected from the group consisting of ACAA2, BIRC5, CD53, CD59, CISD1, COL6A2, FDXR, GTF2IRD2, HADH, HDHD3, IFI27, ISOC2, MRPL27, MYO15B, NFIA, OTOF, PRDX1, RPL26L1, SESTD1, TIMM13, and TXNDC17.

In another preferred embodiment, a feature may include one or more second DEGs in a CD8+ effector memory T cell and the one or more second DEGs may be one or more differentially expressed genes where the genes are selected from the group consisting of COL6A2, POU6F1, MPP7, NFIA, GTF2IRD2, PGAP3, GTF2A2, UBE2L3, GLRX3, PARK7, AHSA1, PGAM1, ACAT2, ACAA2, MRPL27, JPT1, RPA3, CHEK1, FDXR, SESTD1, CHI3L2, and TPX2. The one or more second DEGs may be 1-5, 6-10, 11-15, or 16-22 differentially expressed genes, where the genes are selected from the group consisting of COL6A2, POU6F1, MPP7, NFIA, GTF2IRD2, PGAP3, GTF2A2, UBE2L3, GLRX3, PARK7, AHSA1, PGAM1, ACAT2, ACAA2, MRPL27, JPT1, RPA3, CHEK1, FDXR, SESTD1, CHI3L2, and TPX2.

In some embodiments, a feature may include one or more second DEGs in a CD8+ effector memory T cell. The one or more second DEGs may be one or more differentially expressed genes where the genes are selected from the group consisting of CD278, CD38, CD39, CD49B, CD86, and HLA-DR. The one or more second DEGs may be 1, 2, 3, 4, 5, or 6 differentially expressed genes, where the genes are selected from the group consisting of CD278, CD38, CD39, CD49B, CD86, and HLA-DR.

In some embodiments, a feature may include one or more second DEGs in a CD4+ effector memory T cell (T4em) and the one or more second DEGs may be one or more differentially expressed genes where the genes are selected from the group consisting of ACAD9 and/or TK1. In some embodiments, a feature may include one or more second DEGs in a CD45RA+ effector memory CD4+ T cell (T4ra) and the one or more second DEGs may be one or more differentially expressed DFNB31 gene.

In some embodiments, the second DEG may be located a predetermined first distance from a differentially accessible region (DAR). The second DAR may be detected using a whole genome approach, such as ATAC-Seq. Alternatively, the second DEG may be detected using a targeted approach, such as RT-PCR. In some embodiments, the predetermined second distance is less than 300,000; 250,000; 200,000; 150,000; 100,000; 75,000; 50,000; or 25,000 nucleotides.

In some embodiments, a feature comprises a differentially expressed protein (DEP) in an immune cell subclass. The immune cell subclass may be CD56-high natural killer cells (NKhi), T8em, CD4+ effector memory cells (T4em), naïve CD8+ T cells (T4nv), CD45RA+ effector memory CD8+ T cells (T8ra), or gammadelta T cells (gadT). Protein expression can be measured using any method known to one of ordinary skill in the art.

In some embodiments, the DEP is a protein that is expressed in a subject at a level that is significantly different from the level that it is expressed in a control non-progressor. The DEP may be expressed at a level that is different by a predetermined seventh amount than the expression of the DEP in a control subject (non-progressor). The DEP may be expressed at a level that is at least about 1.5, 1.75, 2, 2.25, 2.5, 2.75, or 3-fold greater or less than the expression of the DEP in a control non-progressor. In some embodiments, the predetermined seventh amount is a value equal to or greater in magnitude to the log 2FC (log 2 fold change) for each DEP shown in Table 6.

TABLE 6
DEPs for immune cell subtypes with false discovery rates and
fold change in expression, shown as log2 fold change (log2FC)

| protein | subset | FDR | log2FC |
|---|---|---|---|
| CD161 | NKhi | 0.04820366 | −0.523465338 |
| CCR10 | T4em | 0.077556464 | 0.627917855 |
| CD229 | T4em | 0.077556464 | 0.259769868 |
| CD86 | T4em | 0.077556464 | 0.875500087 |
| CD94 | T4nv | 0.048776296 | 0.853546907 |
| CD278 | T8em | 0.023830081 | 0.419601584 |
| CD38 | T8em | 0.000485986 | 0.923268425 |
| CD39 | T8em | 0.049879738 | 0.964512553 |
| CD49B | T8em | 0.023830081 | 0.776203879 |
| CD86 | T8em | 0.000485986 | 1.29112242 |
| HLA-DR | T8em | 0.072531919 | 0.479575611 |
| CD30 | T8ra | 0.002192003 | −1.296896039 |
| CD134 | gadT | 0.029993196 | −0.996233536 |
| CD30 | gadT | 0.029993196 | −0.976360161 |

In some embodiments, a feature comprises elevated chromatin accessibility in the MoIn. The elevated chromatin accessibility may be a number of DARs in a subject MoIn that is greater than a number of DARs in a control non-progressor MoIn. The elevated chromatin accessibility may be a number of DARs in a subject MoIn that is greater than the mean number of DARs in control non-progressor MoINs. In some embodiments, the elevated chromatin accessibility number is a number of DARs that is greater than a predetermined number of DARs. The predetermined number of DARs may be 50, 100, 200, 300, 400, 500, 600, 700, 800, 900, or 1000 DARs detected in a subject MoIn.

In some embodiments, the method for determining a signature for identifying a subject at risk of a severe reaction to a SARS-CoV virus comprises:
(i) identifying a subject who has tested positive for the SARS-CoV virus;
(ii) obtaining a blood sample from the subject;
(iii) identifying a cell from the blood sample of the subject, wherein the cell is an intermediate monocyte (MoIn), non-classical monocyte (MoNC), classical monocyte (MoCl), and/or CD8+ effector memory T cell;
(iv) performing an assay on the cell to detect a feature comprising:
   (a) a first differentially expressed gene (DEG) in the MoIn, MoNC, or MoCl
   (b) a second DEG in the CD8+ effector memory T cell, and/or
   (c) elevated chromatin accessibility in the MoIn;
wherein the signature comprises the feature.

In some embodiments, the signature for identifying a subject at risk of a severe reaction to a SARS-CoV virus comprises a feature selected from the group consisting of:
(i) elevated serum concentration of a first cytokine comprising G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα;
(ii) elevated ratio of IL-6 to a second cytokine comprising G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα;
(iii) reduced ratio of an intermediate monocyte subclass (MoIn) and/or non-classical monocyte subclass (MoNC) to total monocytes
(iv) a first differentially expressed gene (DEG) in a monocyte subclass;
(v) a second DEG in a CD8+ effector memory T cell; and
(vi) elevated chromatin accessibility in the MoIn
wherein the feature is determined from a blood sample of a subject who has tested positive for the SARS-CoV virus.

In some embodiments, the present technology includes a method for identifying a subject at risk of a severe reaction to a SARS-CoV virus. The method may comprise (1) determining whether the subject has tested positive for a SARS-CoV virus; and (2) determining whether the signature of the present technology is present in the subject. In some aspects, if the signature is present in the subject the subject is at risk of a severe reaction to a SARS-CoV virus.

The present technology includes a kit for use in identifying a subject at risk of a severe reaction to a SARS-CoV virus comprising any of the materials and reagents of the present technology.

### Methods for Treating COVID-19

In some embodiments, the present technology includes methods for treating a SARS-CoV virus. The method may comprise identifying a subject who has tested positive for a SARS-CoV virus, identifying the signature of the present technology in the subject, categorizing the subject as a progressor if the signature is identified in the subject, or a non-progressor if the signature is not identified in the subject; and if the subject is the progressor, providing a SARS-CoV virus therapeutic to the progressor to treat the SARS-CoV virus; otherwise if the subject is the non-progressor, providing a symptomatic treatment.

As used herein “providing” a therapy includes administering, prescribing, or otherwise assigning a treatment to a subject.

As used herein, “treating” or “treatment” of a SARS-CoV virus may refer to reducing or eliminating the amount of SARS-CoV virus in the subject, reducing or eliminating the symptoms caused by the SARS-CoV virus, reducing the length of the SARS-CoV virus disease course, preventing, delaying, or attenuating the development of a severe reaction to a SARS-CoV virus, improving the outcome of the subject, or some combination thereof. Treatment may also mean a prophylactic or preventative treatment of a condition.

As used herein, “reducing” the amount of a SARS-CoV virus may include decreasing the amount of SARS-CoV virus compared to the amount of SARS-CoV virus in the subject before the treatment was administered. Reducing the symptoms caused by the SARS-CoV virus includes decreasing the amount and/or severity of the symptoms caused by the SARS-CoV virus compared to the symptoms before administration of the treatment.

As used herein, “eliminating” the amount of the SARS-CoV virus in the subject includes decreasing the amount of the SARS-CoV virus to an undetectable level. Eliminating the symptoms caused by the SARS-CoV virus includes decreasing the number and severity of symptoms caused by the SARS-CoV virus to a level that is not detectable by the subject. Reducing the length of the SARS-CoV virus disease course includes decreasing the time that it takes to either eliminate the SARS-CoV virus from the subject or eliminate the symptoms caused by the SARS-CoV virus from the subject.

As used herein, “preventing” development of a severe reaction to a SARS-CoV virus includes blocking the development of one or more symptoms or characteristics of a severe reaction to a SARS-CoV virus. Symptoms of a severe reaction to a SARS-CoV virus may include trouble breathing, pain and pressure in the chest, blue lips or face, confusion, an oxygen saturation less than 94%, a ratio of arterial partial pressure of oxygen to fraction of inspired oxygen less than 300 mm Hg, a respiratory rate greater than 30 breaths/min, lung infiltrates greater than 50%, respiratory failure, septic shock, and/or multiple organ dysfunction. Preventing development of a severe reaction to a SARS-CoV virus also includes preventing the need of additional medical intervention beyond the COVID-19 therapy of the present technology. For example, preventing the development of a severe reaction to a SARS-CoV virus includes preventing the need for supplemental oxygen, intravenous fluids, diuretics, anticoagulation, increased doses of medications for pre-existing condition(s), and/or antibiotics.

As used herein, “delaying” development of a severe reaction to a SARS-CoV virus includes preventing the immediate development of one or more symptom and/or characteristic of a severe reaction to a SARS-CoV virus compared to a control subject progressor. “Attenuating” the development of a severe reaction to a SARS-CoV virus includes slowing the development of one or more symptoms and/or characteristics of a severe reaction to a SARS-CoV virus compared to a control subject progressor.

In some embodiments, the method of treating the SARS-CoV virus comprises categorizing the subject as a progressor or non-progressor. The subject may be categorized as a progressor if the signature is detected in the subject. In some embodiments, the subject may be categorized as a progressor if the subject has one or more features of the signature. The subject may be categorized as a progressor if the subject has 1, 2, 3, 4, 5, or 6 features of the signature.

The subject may be categorized as a non-progressor if the signature is not detected in the subject. In some embodiments, the subject may be categorized as a non-progressor if the subject does not have any features of the signature. The subject may be categorized as a non-progressor if the subject has 0, 1, or 2 of the features of the signature.

In some embodiments, the method of treating a SARS-CoV virus comprises stratifying the subject into more than one category of progressor based on the number of features in the subject. The method may include stratifying the subject into levels based on the likelihood that the subject is a progressor. In some embodiments, the method may include stratifying the subject into grade 1, grade 2, or grade 3, where grade 3 is the most likely to progress, grade 2 is the second most likely to progress, and grade 1 is less likely to progress, but is still more likely to progress than a non-progressor. For example, the subject may be stratified into grade 1 if the subject has 1-2 features of the signature; grade 2 if the subject has 3-4 features of the signature; and grade 3 if the subject has 5-6 features of the signature.

In some embodiments, the method comprises administering or providing a SARS-CoV virus therapeutic to the subject if the subject is a progressor. The method may comprise administering or providing a SARS-CoV virus therapeutic to the subject if the subject is stratified into grade 3, grade 2, and/or grade 1.

The SARS-CoV virus therapeutic may be any therapeutic known in the art that is used to treat a SARS-CoV virus, such as COVID-19. For example, the SARS-CoV virus therapeutic may be an antiviral drug, such as remdesivir, Paxlovid, or Lagevrio; or an immune modulator, such as baricitinib or a corticosteroid. In some embodiments, the subject is treated with an IL-6 inhibitor (e.g. tocilizumab), a TNF inhibitor, and/or a monocyte chemotaxis inhibitor (e.g. cenicriviroc).

In some embodiments, the SARS-CoV virus therapeutic may be a targeted therapeutic based on the signature of the subject. For example, if the signature comprises a feature of an elevated DEG or cytokine and the DEG or cytokines is a known target of a drug, the drug may be the targeted therapeutic. In some embodiments, if the signature comprises a feature of an elevated IL-6 cytokine, an IL-6 inhibitor may be administered or provided. In some embodiments, if the signature comprises a feature of an elevated TNF cytokine, a TNF inhibitor may be administered or provided. In some embodiments, if the signature comprises a feature of reduced ratio of an intermediate monocyte subclass (MoIn) and/or non-classical monocyte subclass (MoNC) to total monocytes, a monocyte chemotaxis inhibitor may be administered.

The COVID-19 therapeutic may be administered or provided using dosing and administration schemes known to one of skill in the art. In embodiments where the subject is stratified into a grade based on likelihood of progressing to a severe reaction to a SARS-CoV virus, the COVID-19 therapeutic may be administered at a dose that is proportional to the likelihood of the subject progressing to a severe reaction to a SARS-CoV virus. For example, a subject that is stratified into a high grade may be administered a higher dose of the SARS-CoV virus therapeutic than a subject that is stratified into a lower grade. The COVID-19 therapeutic may be administered in combination with a second therapy, such as a symptomatic treatment or a second SARS-CoV virus therapeutic. In some embodiments, the SARS-CoV virus therapeutic is administered or provided shortly after the subject is identified as a progressor, for example within 1-2 days of identifying the subject as a progressor.

In some embodiments, the method comprises administering or providing a symptomatic treatment if the subject is a non-progressor. As used herein, a “symptomatic treatment” includes any treatment that is used to treat the symptoms of SARS-CoV virus, but not the underlying disease. Symptomatic treatments may include non-prescription over the counter medications that the subject self-administers. For example, a symptomatic treatment may include over the counter NSAIDs, decongestants, throat lozenges, or antihistamines.

As used herein, “an effective amount” refers to an amount of a composition that produces a desired effect. An effective amount of a composition may be used to produce a prophylactic or therapeutic effect in a subject, such as preventing or treating a target condition, alleviating symptoms associated with the condition, or producing a desired physiological effect. In such a case, the effective amount of a composition is a “therapeutically effective amount,” “therapeutically effective concentration,” or “therapeutically effective dose.” The precise effective amount or therapeutically effective amount is an amount of the composition that will yield the most effective results in terms of efficacy of treatment in a given subject or population of cells. This amount will vary depending upon a variety of factors, including, but not limited to, the characteristics of the composition (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the composition is administered alone or in combination with another composition, drug, therapy, or other therapeutic method or modality. One skilled in the clinical and pharmacological arts will be able to determine an effective amount or therapeutically effective amount through routine experimentation, namely, by monitoring a cell's or subject's response to administration of a composition and adjusting the dosage accordingly.

A “clinically effective amount,” “clinically effective concentration,” or “clinically effective dose” refers to a concentration or dose of a treatment that is shown to be effective in clinical trials or is predicted to be effective based on early phase or pre-clinical trials. For additional guidance, see Remington: The Science and Practice of Pharmacy, 21^{st} Edition, Univ. of Sciences in Philadelphia (USIP), Lippincott Williams & Wilkins, Philadelphia, PA, 2005.

In some embodiments, the method of treating a SARS-CoV virus comprises:
(i) identifying a subject who has tested positive for the SARS-CoV virus;
(ii) identifying a signature in the subject, wherein the signature comprises a feature selected from the group consisting of:
   (a) elevated serum concentration of a first cytokine comprising G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα;
   (b) elevated ratio of IL-6 to a second cytokine comprising G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα;
   (c) reduced ratio of an intermediate monocyte subclass (MoIn) and/or non-classical monocyte subclass (MoNC) to total monocytes;
   (d) a first differentially expressed gene (DEG) in a monocyte subclass;
   (e) a second DEG in a CD8+ effector memory T cell; and
   (f) elevated chromatin accessibility in the MoIn;
(iii) categorizing the subject as a progressor if the signature is identified in the subject, or a non-progressor if the signature is not identified in the subject;
(iv) if the subject is the progressor, providing a SARS-CoV virus therapeutic to the progressor to treat SARS-CoV virus; and
(v) if the subject is the non-progressor, providing a symptomatic treatment.

The present technology includes methods of forming a treatment plan comprising identifying a signature of the present technology in a subject that has tested positive for a SARS-CoV virus and providing instructions for administering a SARS-CoV virus therapeutic or symptomatic therapeutic. The treatment plan may include classifying the subject as a progressor or non-progressor based on the signature. The treatment plan may also include stratifying the subject based on the signature. In some embodiments, the treatment plan may include administering a SARS-CoV virus only if the subject is stratified into a high-risk grade. In some aspects, the treatment plan may include administering a SARS-CoV virus therapeutic if the subject is stratified into a high-risk grade and a symptomatic therapeutic or no therapeutic if the subject is stratified into a low-risk grade.

In some embodiments, the treatment plan may include modifying the treatment by identifying the presence of a second signature in the subject after the subject has received a SARS-CoV virus therapeutic (i.e. a first SARS-CoV virus therapeutic). The treatment plan may include terminating the first SARS-CoV virus therapeutic if the second signature is not present in the subject. The treatment plan may include providing a second SARS-CoV virus therapeutic if the second signature is present in the subject.

The present technology includes methods of determining prognosis or risk of progression of a subject who has tested positive for a SARS-CoV virus comprising identifying the signature of the present technology in the subject.

Immune Profiler Platform and Implementation of Learnings from Multi-Omic Analysis

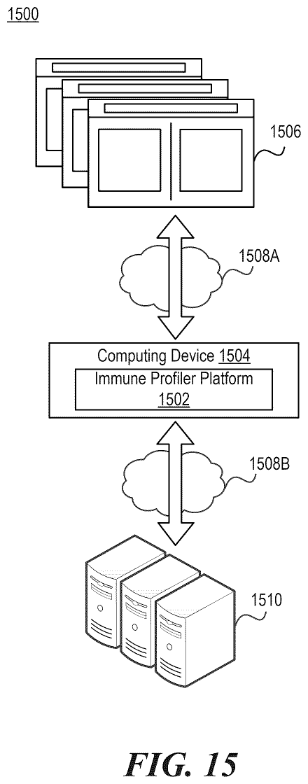

FIG. 15 illustrates a network environment 1500 that includes an Immune Profiler platform 1502 that is executed by a computing device 1504. The Immune Profiler platform 1502 may be used to predict diagnoses for patients as further discussed below, and therefore could also be called a “diagnostic platform.”

An individual (also referred to as a “user”) can interact with the Immune Profiler platform 1502 via interfaces 1506. For example, a healthcare professional may be able to access an interface through which information regarding a subject who has tested positive for the SARS-CoV virus can be reviewed. As another example, a healthcare professional may be able to access an interface through which information regarding subjects prescribed different therapeutic treatments can be reviewed. Examples of healthcare professionals include physicians, nurses, nurse practitioners, and the like. The interfaces 1506 may allow for the review of physiological data, as well as examination of outputs produced by the Immune Profiler platform 1502 and management of preferences. Some interfaces may be configured to facilitate interactions between patients and healthcare professionals, while other interfaces may be configured to serve as informative dashboards for patients or healthcare professionals.

As shown in FIG. 15, the Immune Profiler platform 1502 can reside in a network environment 1500. Thus, the computing device 1504 on which the Immune Profiler platform 1502 resides can be connected to one or more networks 1508A-B. Depending on its nature, the computing device 1504 could be connected to a personal area network (PAN), local area network (LAN), wide area network (WAN), metropolitan area network (MAN), or cellular network. For example, if the computing device 1504 is a computer server, then the computing device 1504 may be accessible to healthcare professionals via respective computing devices that are connected to the Internet via LANs. The data to be examined by the Immune Profiler platform 1502 may be acquired from these respective computing devices, acquired from other computing devices, or generated by the Immune Profiler platform 1502. As an example, assume that the Immune Profiler platform 1502 is tasked—by a healthcare professional that accesses the interfaces 1506 via a laptop computer—with predicting whether a subject is likely to develop a severe reaction to the SARS-CoV virus based on an analysis of cellular, molecular, or multi-omic data that is associated with the subject. That data could be uploaded to the Immune Profiler platform 1502 by the laptop computer, acquired by the Immune Profiler platform 1502 from another computing device (e.g., a storage medium that is accessible via the Internet), or generated by the Immune Profiler platform 1502.

The interfaces 1506 may be accessible via a web browser, desktop application, mobile application, or another form of computer program. For example, a healthcare professional may be able to access interfaces through which information regarding one or more subjects can be reviewed via a web browser. Accordingly, the interfaces 1506 generated by the Immune Profiler platform 1502 may be accessible on various computing devices, including mobile phones, tablet computers, desktop computers, and the like.

Generally, the Immune Profiler platform 1502 is executed—at least partially—by a cloud computing service operated by, for example, Amazon Web Services®, Google Cloud Platform™, or Microsoft Azure®. Thus, the computing device 1504 may be representative of a computer server that is part of a server system 1510. Often, the server system 1510 is comprised of multiple computer servers. These computer servers can include different types of data (e.g., cellular, molecular, or multi-omic data and information regarding subjects, such as name, demographic information, disease classification, treatment regimen, etc.), algorithms for processing incoming data, and other assets. Those skilled in the art will recognize that these data could also be distributed among the server system 1510 and one or more computing devices. As an example, sensitive data that is input by, or related to, subjects being treated by a healthcare system may be stored on, and processed by, computing devices managed by the healthcare system for security or privacy purposes. Alternatively, sensitive data could be locally obfuscated (e.g., encrypted)—on a computing device managed by the healthcare system—prior to transmittal to the server system 1510 for analysis by the Immune Profiler platform 1502.

Components of the Immune Profiler platform 1502 could also be hosted locally. That is, part of the Immune Profiler platform 1502 may reside on the computing device used to access one of the interfaces 1506. For example, the Immune Profiler platform 1502 may be at least partially embodied as a desktop application executing on a laptop computer accessible to a healthcare professional. Note, however, that the desktop application may be communicatively connected to the server system 1510 on which other components of the Immune Profiler platform 1502 are hosted.

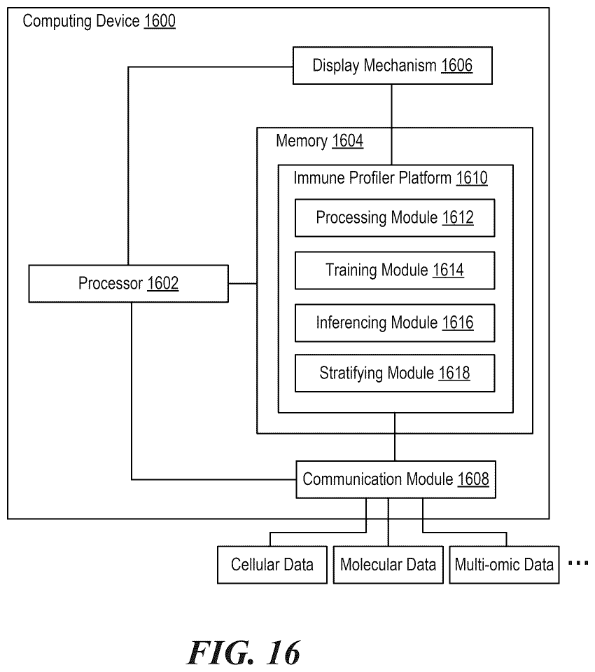

FIG. 16 illustrates an example of a computing device 1600 that is able to implement an Immune Profiler platform 1610. As further discussed below, the Immune Profiler platform 1610 may be able to develop a machine learnt model for predicting likelihood of severe SARS-CoV reaction, as well as apply the machine learnt model for the purpose of prioritizing subjects for examination, stratifying subjects among different treatments, etc. As shown in FIG. 16, the computing device 1600 can include a processor 1602, memory 1604, display mechanism 1606, and communication module 1608. Each of these components is discussed in greater detail below.

Those skilled in the art will recognize that different combinations of these components may be present depending on the nature of the computing device 1600. For example, if the computing device 1600 is a computer server that is part of a server system (e.g., server system 1510 of FIG. 15), then the computing device 1600 may not include the display mechanism 1606. Conversely, if the computing device 1600 is a laptop computer, then the computing device 1600 can include the display mechanism 1606.

In some embodiments, the Immune Profiler platform 1610 is responsible for generating the cellular data, molecular data, or multi-omic data to be examined, for example, through an analysis of data generated via an assay. In embodiments where the Immune Profiler platform 1610 generates the data of interest, the data of interest is generally examined—for the purpose of determining whether the corresponding subject is likely to develop severe symptoms—immediately after being generated. In other embodiments, the Immune Profiler platform 1610 acquires—via the communication module 1608—the cellular data, molecular data, or multi-omic data from a source external to the computing device 1600. In such embodiments, the data of interest need not necessarily be examined immediately after being generated. For example, a healthcare system may routinely generate cellular data, molecular data, or multi-omic data for subjects as part of its routine examination, but that data may only be examined further if one of the subjects tests positive for the SARS-CoV virus or reports certain symptoms stemming from testing positive for the SARS-CoV virus.

The processor 1602 can have generic characteristics similar to general-purpose processors, or the processor 1602 may be an application-specific integrated circuit (ASIC) that provides control functions to the computing device 1600. As shown in FIG. 16, the processor 1602 can be coupled to all components of the computing device 1600, either directly or indirectly, for communication purposes.

The memory 1604 can be comprised of any suitable type of storage medium, such as static random-access memory (SRAM), dynamic random-access memory (DRAM), electrically erasable programmable read-only memory (EEPROM), flash memory, or registers. In addition to storing instructions that can be executed by the processor 1602, the memory 1604 can also store data generated by the processor 1602 (e.g., when executing the modules of the Immune Profiler platform 1610). Note that the memory 1604 is merely an abstract representation of a storage environment. The memory 1604 could be comprised of actual integrated circuits (also called “chips”).

The display mechanism 1606 can be any mechanism that is operable to visually convey information to a user. For example, the display mechanism 1606 can be a panel that includes light-emitting diodes (LEDs), organic LEDs, liquid crystal elements, or electrophoretic elements. Outputs produced by the Immune Profiler platform 1610 (e.g., through execution of its modules) can be posted to the display mechanism 1606 for review by a user of the computing device 1600.

The communication module 1608 may be responsible for managing communications external to the computing device 1600. The communication module 1608 can be wireless communication circuitry that is able to establish wireless communication channels with other computing devices. Examples of wireless communication circuitry include 2.4 gigahertz (GHz) and 5 GHz chipsets compatible with Institute of Electrical and Electronics Engineers (IEEE) 802.11—also referred to as “Wi-Fi chipsets.” Alternatively, the communication module 1608 may be representative of a chipset configured for Bluetooth®, Zigbee®, near-field communication (NFC), or another short-range wireless communication technology. Some computing devices—like mobile phones, tablet computers, and the like—are able to wirelessly communicate via separate channels, while other computing devices—like computer servers—tend to wirelessly communicate via a single channel. Accordingly, the communication module 1608 may be one of multiple communication modules implemented in the computing device 1600, or the communication module 1608 may be the only communication module implemented in the computing device 1600.

The nature, number, and type of communication channels established by the computing device 1600—and more specifically, the communication module 1608—can depend on (i) the sources from which data is received by the Immune Profiler platform 1610 and (ii) the destinations to which data is transmitted by the Immune Profiler platform 1610. Assume, for example, that the Immune Profiler platform 1610 resides on a laptop computer in the form of a desktop application. In such embodiments, the communication module 1608 can communicate with a source external to the computing device 1600 from which to obtain cellular data, molecular data, or multi-omic data. Moreover, the communication module 1608 may communicate with a server system (e.g., server system 1510 of FIG. 15) to which analyses of the data—or the data itself—are transmitted.

For convenience, the Immune Profiler platform 1610 is referred to as a computer program that resides within the memory 1604. However, the Immune Profiler platform 1610 could be comprised of software, firmware, or hardware that is implemented in, or accessible to, the computing device 1600. In accordance with embodiments described herein, the Immune Profiler platform 1610 can include a processing module 1612, training module 1614, inferencing module 1616, and stratifying module 1618. These modules could be integral parts of the Immune Profiler platform 1610, or these modules could be logically separate from the Immune Profiler platform 1610 but operate “alongside” it. Together, these modules enable the Immune Profiler platform 1610 to establish the likelihood that a subject will experience severe symptoms from the SARS-CoV virus. Embodiments of the Immune Profiler platform 1610 could also include other modules not shown in FIG. 16, such as a graphical user interface (“GUI”) module that is responsible for generating the interfaces that are viewable via the display mechanism 1606.

The processing module 1612 can process data that is obtained by the Immune Profiler platform 1610 into a format that is suitable for the other modules. Assume, for example, that the Immune Profiler platform 1610 acquires—via the communication module 1608—cellular data, molecular data, or multi-omic data from a source external to the computing device 1600, like a network-accessible storage. The processing module 1612 may apply operations to the data in preparation for analysis by the other modules of the Immune Profiler platform 1610. For example, if a machine learnt model is to be applied to the data, the processing module 1612 may filter the data to lessen the volume that is fed into the machine learnt model or reformat the data into a format that can be more readily handled by the machine learnt model. These steps may allow for greater efficiencies (e.g., in terms of computational resources). As another example, the processing module 1612 may be responsible for concatenating data acquired from different sources, but associated with the same subject, into a data structure. For example, the processing module 1612 may populate a data structure with information associated with a subject, such as age, gender, and comorbidities, and cellular data, molecular data, or multi-omic data.

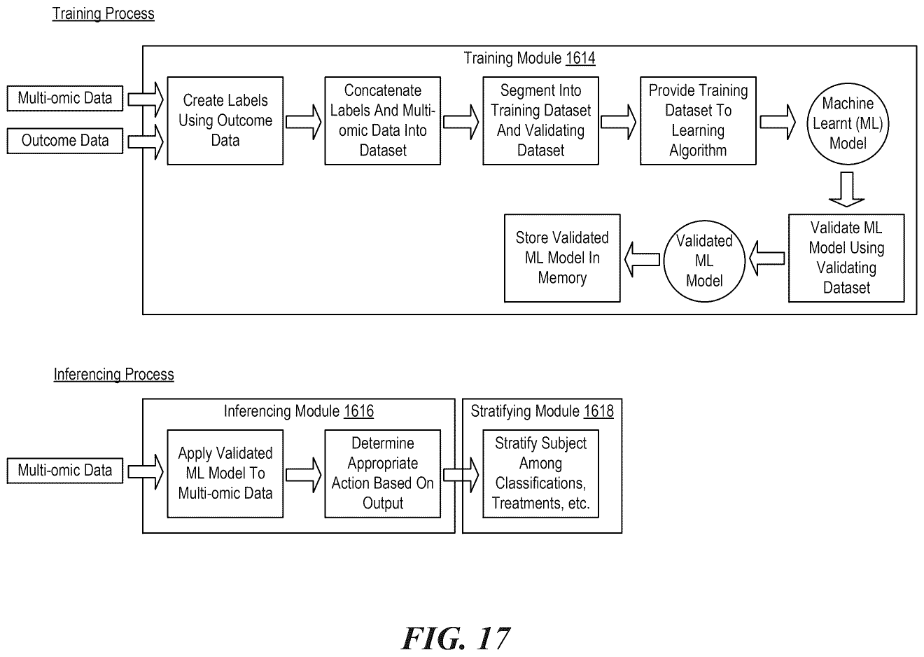

The training module 1614 may be responsible for training a machine learnt model to predict the likelihood that subjects will experience severe symptoms from the SARS-CoV virus from an analysis of cellular data, molecular data, or multi-omic data. Note that the term “machine learnt model” may be used interchangeably with the terms “machine learning model” or simply “model.” At a high level, the process of training a machine learnt model involves providing a machine learning algorithm (or simply “learning algorithm”) with a training dataset from which to learn relationships and another dataset—commonly called a “validating dataset”—from which to validate the learned relationships. The learning algorithm tries to discover patterns in the training dataset that relate attributes and labels and then outputs the machine learnt model that captures these patterns. Consider, for example, a scenario in which the Immune Profiler platform 1610 is able to access (i) multi-omic data for various subjects that have tested positive for the SARS-CoV virus and (ii) outcome data that indicates how each of the various subjects responded to testing positive for the SARS-CoV virus, as shown in FIG. 17. The outcome data could be binary in nature, specifying whether each subject survived for a predetermined amount of time (e.g., 60, 90, or 120 days), whether each subject experienced severe symptoms, whether each subject was ultimately diagnosed with “long COVID,” etc. Alternatively, the outcome data could be non-binary in nature, specifying the symptoms each subject experienced, the degree to which the symptoms impacted quality of life, etc.

Regardless of its form, the training module 1614 may concatenate the outcome data with the multi-omic data into a dataset, with information gleaned from the outcome data being used to create labels that are associated with (e.g., appended to) corresponding portions of the multi-omic data. Assume, for example, that the training module 1614 is tasked with creating a machine learnt model that is able to predict the likelihood that a subject will experience severe symptoms from the SARS-CoV virus. In such a scenario, the training module 1614 can segment the multi-omic data, such that a separate portion of the multi-omic data is identified for each of the subjects, and then label each portion of the multi-omic data based on an analysis of the outcome data associated with a corresponding one of the subjects. This dataset—with multi-omic data and labels created based on the outcome data—can then be segmented into two datasets, namely, a training dataset and a validating dataset. Generally, the training dataset is larger than the validating dataset, with the training dataset being about 60-90 percent (and preferably 70-80 percent) of the size of the original dataset.

In order for the learning algorithm to create an accurate model artifact, the training dataset must contain the answer to be predicted—commonly called the “target.” In this scenario, the labels can be used to indicate which portions of multi-omic data relate to severe outcomes from the SARS-CoV virus. Through the learning process, the learning algorithm can discover patterns in the training dataset that map attributes of the multi-omic data to the target, and the learning algorithm can output the machine learnt model that captures these patterns. In some embodiments, the learning process is completed in a semi-supervised manner with assistance. For example, the individual responsible for training the machine learnt model may already have insight into features that may be collectively representative of a signature for higher likelihood of experiencing a severe outcome. That knowledge could be supplied to the learning algorithm, in an effort to have the learning algorithm confirm whether those features are actually indicators of more severe outcomes. In other embodiments, the learning process is completed in an unsupervised manner, such that the learning algorithm is allowed to identify those features that are collectively representative of the signature on its own without assistance.

Evaluating whether the machine learnt model performs as expected is an important part of the training process. The multi-omic data to which the machine learnt model is applied during the inferencing stage has an unknown target, and therefore it is important to check the accuracy of the machine learnt model on data for which the target is known. That's where the validating dataset can be used, namely, as a means of assessing accuracy as a proxy for predictive accuracy on future multi-omic data.

To properly evaluate the machine learnt model, the learning module 1614 can hold onto a portion of the original dataset—namely, the validating dataset—that is used only for validation. Evaluating the predictive accuracy of the machine learnt model with the same training dataset supplied to the learning algorithm is not useful, as the machine learnt model would be rewarded for remembering the training dataset rather than learning to generalize from it. Specifically, portions of multi-omic data in the validating dataset can be supplied to the machine learnt model that produces, as output, predictions. These predictions and then be compared to the labels created for those portions of multi-omic data in order to establish a metric that indicates how well the machine learnt model performs. The metric may be representative of an indication of how well the predictions output by the machine learnt model match the labels. In response to a determination that the metric exceeds a threshold—indicating that performance of the machine learnt model is sufficient—the training module 1614 can store the now-validated machine learnt model in memory (e.g., memory 1604 of FIG. 16 or memory external to computing device 1600 of FIG. 16). In response to a determination that the metric does not exceed the threshold—indicating that performance of the machine learnt model is not sufficient—the training module 1614 may perform some other action. For example, the training module 1614 may initiate a retraining operation in which aspects (e.g., weights) of the machine learnt model are adjusted to see whether performance can be improved. As another example, the training module 1614 may restart the training process with different multi-omic data or more multi-omic data.

Thereafter, the Immune Profiler platform 1610 may be tasked with implementing the machine learnt model. Assume, for example, that the Immune Profiler platform 1610 obtains multi-omic data for a subject who has tested positive for the SARS-CoV virus. In such a scenario, the inferencing module 1616 can apply the machine learnt model to the multi-omic data to produce an output. At a high level, this output is representative of a label that is predicted by the machine learnt model through an analysis of the multi-omic data. Thereafter, the inferencing module 1616 can determine an appropriate action based on the output. For example, if the output indicates that the subject is at no risk—or little risk—of severe symptoms from the SARS-CoV virus, then no further action may be necessary. However, if the output indicates that the subject is at moderate or high risk of severe symptoms, then the inferencing module 1616 may take further action. For example, the inferencing module 1616 may store an indication of the output in a digital profile that is representative of an electronic health record. As another example, the inferencing module 1616 may cause a notification to be generated and presented to a healthcare professional that can provide care to the subject. As another example, the inferencing module 1616 may identify whether the subject is a good candidate for a given treatment regimen—or treatment more generally—based on an analysis of information that is available for the subject. For instance, the inferencing module 1616 may attempt to establish whether the subject is a good candidate for an antiviral drug or immune modulator based on age, gender, weight, comorbidities, etc. Note that the stratifying module may also, or alternatively, be responsible for taking action based on the output of the machine learnt model. For example, based on the output of the machine learnt model, the stratifying module 1618 may stratify the subject among different classifications (e.g., low risk, moderate risk, high risk), different urgencies (e.g., low concern, moderate concern, urgent concern), different treatment regimens (e.g., antiviral drug, immune modulator, rest only), etc. Such an appropriate to autonomously stratifying subjects allows those subjects to be managed in a more efficient manner, with resources being committed to those subjects that are most at risk.

While FIG. 17 illustrates exemplary training and inferencing processes that use multi-omic data, the processes may be largely similar if cellular data or molecular data is used instead of, or in addition to, multi-omic data.

Several examples of machine leant models are provided below.

Binary Classification Models: The output of a binary classification model specifies one of two classes. An example of such a machine learnt model is one that when applied to multi-omic data, determines whether the corresponding subject will or will not develop severe symptoms from the SARS-CoV virus.

Non-Binary Classification Models: The output of a non-binary classification model specifies one of at least three classes. An example of such a machine learnt model is one that when applied to multi-omic data, specifies whether the corresponding subject has a low risk, moderate risk, or high risk of developing severe symptoms from the SARS-CoV virus.

Regression Models: The output of a regression model is generally a single number (e.g., 50 percent) or an interval of numbers (e.g., 40-60 percent). An example of such a machine learnt model is one that when applied to multi-omic data, estimates the probability that the corresponding subject will develop severe symptoms from the SARS-CoV virus. One of the benefits of a regression model is that it can provide context that is helpful to healthcare professionals. For example, different action may be taken if the machine learnt model indicates that the subject is 70-90 percent likely to develop severe symptoms versus 50-70 percent likely to develop severe symptoms.

The nature of the machine learnt model may not only affect how its outputs are used by the Immune Profiler platform 1610, but also how training, validating, and/or inferencing are performed. To train a binary classification model, the training module 1614 may use the learning algorithm known as logistic regression. To train a multiclass classification model, the training module 1614 may use the learning algorithm known as multinomial logistic regression. Other learning algorithms that could be used for classification include (i) the Decision Tree Classifier that uses a decision tree, as a predictive model, to go from observations to conclusions about the target, (ii) the Random Forest Classifier that implements ensemble learning by constructing various decision trees during the training stage and then making a prediction based on the outputs of those decision trees during the inferencing stage, (iii) the XGBoost Classifier, (iv) the Naïve Bayes algorithm that is based on Bayes theorem, (v) support-vector machines (SVMs) that, during the training stage, maps examples to points in space to maximize the width of the gap between different classifications and then, during the inferencing stage, maps new examples into that same space to formulate a prediction, and (vi) neural networks that learn by adjusting weights assigned to nodes across different layers. To train a regression model, the training module 1614 may use the learning algorithm known as linear regression.

The nature of the machine learnt model—and the nature of the learning algorithm and underlying model architecture—may depend on the expectations of the Immune Profiler platform 1610 and computational resources available to the Immune Profiler platform 1610. Neural networks, especially deep ones with more than three layers of nodes, tend to require a significant amount of computational resources during the training stage (and a decent amount of computational resources during the inferencing stage). Performance is generally quite good, however. Accordingly, the training module 1614 may opt to train and implement a neural network so long as sufficient computational resources are available.

Several of the aforementioned learning algorithms—and the corresponding machine learnt models produced as output—perform more efficiently when handling structured data. The exact structure may be less important than the fact that the data can be organized, for example, by the processing module 1612, into a consistent structure. Simply put, the consistent structure can ease the manipulation of the data, making it less “costly” to train and implement the machine learnt model.

### EXAMPLES

From the foregoing, it will be appreciated that specific embodiments of the invention have been described herein for purposes of illustration, but that various modifications may be made without deviating from the scope of the invention. Accordingly, the invention is not limited except as by the appended claims.

### Example 1: Methods

### Study Design

The COVID Progression Retrospective (CPR) study is a retrospective cross-sectional study of existing datasets from Progressors and Non-progressors from two studies: COVID-19 Immune Response Study (Cove) and Predictors of Severe COVID-19 Outcomes (PRESCO).

Cove is a decentralized, prospective study collecting biological measurements and clinical and epidemiological data in participants confirmed positive for SARS-CoV-2 at the time of COVID testing, with the aim of characterizing molecular signatures associated with COVID-19 disease progression over 28 days. Adults testing positive for COVID-19 from the Baseline COVID-19 Testing Program or other partner testing programs and meeting the eligibility criteria were invited to enroll in the Cove study. Eligible participants were adults that 1) were 18 years old or older, 2) were U.S. residents, 3) tested positive for COVID-19 within the past 5 days, 4) were willing and able to provide informed consent, and 5) were willing and able to complete all study procedures. Additionally, participants were excluded if they 1) were pregnant or planning to become pregnant within the next 30 days, 2) had prior history of HIV seropositivity or cancer, 3) were undergoing treatment with immunosuppressants, 4) had known chronic or acute infections other than SARS-CoV-2, or 5) received any dose of a COVID-19 vaccine. A total of 115 participants were enrolled between January and May of 2021 across 5 states (NJ, CA, PA, TX, and NY).

Longitudinal clinical data and biospecimens were collected at up to 3 home visits and through daily electronic patient-reported outcomes (ePROs) following study enrollment, with home visits occurring on Days 3, 5, and 7 and an outcome survey on Day 28. Written informed consent was obtained from all participants or their legally authorized representatives before study-related procedures were performed.

PRESCO (clinicaltrials.gov NCT04388813) is a multi-center, prospective, 3-month cohort study designed to identify clinical and molecular signatures associated with progression to severe COVID-19. Adults with laboratory-test confirmed acute SARS-CoV-2 infection (RT-PCR or antigen testing) who received care at eight sites (The University of Arizona, Cedars-Sinai Medical Center, University of Illinois at Chicago, Rush University Medical Center, Weill Cornell Medical College, University of Texas Southwestern Medical Center, Baylor College of Medicine, and Inova Health Care Services) were invited to participate. A total of 494 patients were enrolled between May 2020 and June 2021 at both ambulatory and hospital-based locations.

Longitudinal clinical data and biospecimens were collected at up to five visits during SARS-CoV-2 infection and recovery: (1) enrollment during initial hospital presentation, and if occurred, (2) two days after hospitalization; (3) on the day of admission to intensive care unit (ICU); (4) the day of hospital discharge; and (5) approximately 3 months after hospital presentation. The study was approved by a central Western Institutional Review Board (Protocol #20201016) and at each of the eight sites. Written informed consent was obtained from all participants or their legally authorized representatives before study-related procedures were performed.

All datasets from participants enrolled in the Cove study were eligible to be included in CPR. For a participant dataset from PRESCO to be included, the participant must have tested positive for SARS-CoV-2 within 5 days prior to or after enrollment in the PRESCO study. Additionally, datasets from participants that met the following criteria were excluded from the CPR study: 1) prior history of HIV seropositivity or cancer, 2) dexamethasone treatment before visit 1, 3) any immunosuppressive therapy within the prior 14 days, 4) known chronic or acute infection other than SARS-CoV-2, and 5) received any dose of COVID-19 vaccine. In total, 83 participant datasets from PRESCO were combined with the 115 from Cove for CPR.

### Clinical Assessment of COVID-19 Progression

COVID-19 progression (i.e. the disease progression of a “progressor”) was defined as 1) hospitalization for a reason either directly or indirectly (e.g., worsening of a pre-existing condition) related to SARS-CoV-2 or 2) outpatient treatment received for a condition either directly or indirectly related to SARS-CoV-2. Outpatient treatment definition excluded any over-the-counter (OTC) medications such as antipyretics, antitussives, and analgesics, but included treatments such as supplemental oxygen, intravenous fluids, diuretics, anticoagulation, increase in dose of medications for pre-existing condition(s), and antibiotics. Progression outcome was adjudicated by an independent panel of 3 clinicians with experience and expertise in respiratory viral infections and the conduct of clinical trials. Non-progressor patients represented individuals in the study that did not meet the progression criteria within 28 days. In some cases where the adjudication panel did not have enough information, no progression outcome was assigned.

All comparisons within this work were between Progressors and Non-progressors, unless otherwise specified. Of the 198 participant datasets eligible for the CPR study, 14 were excluded from analysis due to the adjudication panel having insufficient information to clearly determine a progression outcome. Additionally, 22 participant datasets were excluded from analysis because they: 1) did not meet requirements for study completion, which was defined as completing the 28-day study window regardless of progression outcome, or progressing to clinically significant COVID-19, 2) had evidence of some immunomodulating treatments prior to biospecimen collection, or 3) did not report any symptoms prior to or throughout the study. Lastly, 4 additional participant datasets were excluded from analysis due to lack of biospecimen availability and/or provenance. In total, 162 participant datasets were eligible for descriptive analyses of demographic and clinical variables, and 158 participant datasets were included for the molecular analyses of the CPR study.

### Multi-Omic Analysis

An Immune Profiler platform was employed to conduct multi-omic analysis. This platform begins with the isolation of 25 immune cell subsets (5 myeloid cell subsets, 7 B cell subsets, 10 T cell subsets, 2 NK cell subsets, and a bulk peripheral blood mononuclear cell (PBMC) sample) from a starting material of approximately 10 million cryopreserved PBMCs per individual. Genome-wide chromatin accessibility (ATAC-seq) and transcriptome-wide gene expression (RNA-seq) is measured for each of the 25 subsets, and Targeted Protein Estimation by sequencing (TaPE-seq) is performed for the 12 subsets in the T and NK panel (FIG. 6). Additionally, whole genome sequencing data are generated as permitted by participant consent.

For florescence-activated cell sorting (FACS), frozen cryovials of PBMCs in liquid nitrogen were thawed in a 37° C. water bath and transferred to a 1.5 mL Eppendorf tube. 500 μL of warmed R10 media was added to the 1.5 mL tube and let sit for 2 minutes to come to equilibrium. The cells were then centrifuged for 5 min at 500×g, 25° C. The cell pellet was resuspended with 1 mL of warm FACS buffer and the cells were counted. The cells were then centrifuged again for 5 minutes at 500×g, 4° C. The cell pellet was resuspended in the 50 μL of FACS buffer with 5 μl of BD Human Fc Block and incubated for 5 minutes. 50 μL of staining cocktail was added per 10 million cells counted for the respective flow cytometry panels to be analyzed (T cell, B cell, myeloid panel) and incubated for 15 minutes at 4° C. and in the dark. Cells for B cell panels and myeloid panels were washed in FACS buffer, resuspended in a final volume of 500 μL FACS buffer, and passed through a 35 μm cell strainer cap. 5 μL of 7-AAD live/dead dye was added to the cells before sorting. Cells for T cell panels were washed in FACS buffer, stained with 250 μL of OligoAb cocktail (Tables 1 and 2), incubated for an additional 15 minutes at 4° C. and in the dark, re-washed in FACS buffer, resuspended in a final volume of 500 μL FACS buffer, and passed through a 35-μm cell strainer cap. 5 μL of 7AAD live dead dye was added to the cells before sorting. Stained samples were sorted on a FACSAria Fusion (BD Biosciences, San Jose, CA). Using FACSDiva v8.0.1 software, the samples were gated first by forward and side scatter properties, then FSC-H vs FSC-A for singlet discrimination, and finally, with their respective markers for each cell type (FIG. 6). For each cell type of interest, 800-10,000 cells per sample were sorted into the tagmentation buffer. Additionally, a minimum of 500,000 cells were recovered from either the PBMCs or a separate buffy coat aliquot for whole genome sequencing (WGS).

RNA was separated from the other components, including tagmented DNA and antigens bound by barcoded antibodies in the cell-containing samples, for further analysis. Biotin-OligodTVN beads were added to each sample, mixed, and beads captured using a magnet. The supernatant was aspirated and the plate was removed from the magnet. The beads were resuspended with the lysed cells for 30 minutes at 25° C. with orbital mixing at 1500 rpm using an Eppendorf Thermomixer (Eppendorf, Hamburg, Germany). Samples were centrifuged and placed on a magnet, and the supernatant was transferred into a new plate for subsequent ATAC-seq and TaPE-seq processing. The plate containing the RNA samples was immediately processed through the RNA-seq workflow.

Assay for transposase-accessible chromatin with sequencing (ATAC-seq) was performed according to M. H. Guo et al. 23 Genome Biol. 1-23 (2022), with the exception that barcoded oligos from proteins bound by the OligoAb cocktail (Tables 1 and 2) were simultaneously captured in the supernatant during the above hybridization and separation step for protein estimation using TaPE-seq.

RNA-seq was performed using a switching mechanism at the end of the 5′ end of the RNA template (SMART-Seq2)-based procedure optimized for use on hundreds of cells, as disclosed in U.S. Pat. No. 11,352,714, disclosed herein in its entirety.

RNA-seq libraries and libraries containing both ATAC-seq and TaPE-seq were quantified by qPCR using the KAPA Library Quantification Kit (Complete kit, Universal) (F. Hoffmann-La Roche A G, Basel, Switzerland) on the CFX384 Touch™ Real-Time PCR Detection System (Bio-Rad Laboratories Inc, Hercules, CA, USA). Libraries were pooled and sequenced utilizing a two-pass approach: pools of ATAC-seq and TaPE-seq libraries and pools of RNA-seq libraries were pooled in equimolar ratios and sequenced across a single lane of a flow cell each; libraries were re-pooled after this first-pass sequencing accounting for both their qPCR quantification and the number of first-pass sequencing reads achieved to obtain a more even read distribution across the pool during second-pass sequencing. Libraries were sequenced to total target depths of 10,000,000 reads for RNA-seq, 20,000,000 reads for ATAC-seq, and 50,000 reads for TaPE-seq on the Illumina NovaSeq 6000 platform (Illumina Inc, San Diego, CA, USA), with v1.5 chemistry kits generating paired-end (2×150 bp) reads.

Whole gene sequencing (WGS) was performed on isolated cells using a PCR-free procedure. Briefly, a total of 350 ng of gDNA from each sample based on Quant-iT Picogreen quantification (Thermo Fisher Scientific, Waltham, MA, USA) was mechanically fragmented to a target size between 350 and 400 base pairs (bp) on the Covaris LE220 focused ultrasonicator (Covaris, Woburn, MA, USA). The sheared gDNA underwent size selection using AMPure XP beads (Beckman Coulter, Brea, CA, USA) to tighten the size distribution of the gDNA fragments. The size selected gDNA was end repaired, A-tailed, and adapter-ligated using the KAPA PCR-free Hyper Prep Kit in combination with KAPA Unique Dual-Indexed Adapters (F. Hoffmann-La Roche A G, Basel, Switzerland). Adapter-ligated libraries were purified by AMPure XP bead cleanup. Library yields were assessed by qPCR using the KAPA Library Quantification Kit (Complete kit, Universal) (F. Hoffmann-La Roche A G, Basel, Switzerland) on the CFX384 Touch™ Real-Time PCR Detection System (Bio-Rad Laboratories Inc, Hercules, CA, USA). Dual-indexed libraries were subsequently pooled and sequenced to a target depth of 30× coverage on the Illumina NovaSeq 6000 platform (Illumina Inc, San Diego, CA, USA), with v1.5 chemistry kits generating paired-end (2×150 bp) reads.

### Chromatin Contact Maps

A cutoff of 0.05 counts per million was applied to determine the presence of chromatin contact. These chromatin contact maps for each cell subset specific functional genomics data are measured by Hi-C with chromatin immunoprecipitation (HiChIP).

### Quantification of Viral Load

Viral load was quantified using droplet digital PCR (ddPCR) from mid-turbinate swabs collected at each visit. Extraction of SARS-CoV-2 RNA was performed using the MagMax Viral/Pathogen Nucleic Acid Isolation kit following manufacturer's instructions, and ddPCR was performed according to M. K. Wolfe et al., 6 mSystems e00829-21 (2021). In addition to quantifying the N and ORF1a SARS-CoV-2 genes, RPP30 was quantified as a positive control for swabbing using publicly available sequences.

### Quantification of Plasma Cytokines

Forty-seven cytokines (sCD40L, EGF, Eotaxin, FGF-2, Flt-3 ligand, Fractalkine, G-CSF, GM-CSF, GROα, IFNα2, IFNγ, IL-1α, IL-1β, IL-1ra, IL-2, IL-3, IL-4, IL-5, IL-6, IL-7, IL-8, IL-9, IL-10, IL-12p40, IL-12p70, IL-13, IL-15, IL-17A, IL-17E/IL-25, IL-17F, IL-18, IL-22, IL-27, IP-10, MCP-1, MCP-3, M-CSF, MDC, MIG, MIP-1α, MIP-1β, PDGF-AA, PDGF-AB/BB, TGFα, TNF (TNFα), LTA (TNFβ), and VEGF-A) were quantified from plasma samples using the MILLIPLEX MAP Human Cytokine/Chemokine/Growth Factor Panel A on a Luminex® FLEXMAP 3D instrument according to manufacturer's instructions. For each cytokine measured, values lying outside of the standard curve were imputed to the nearest standard concentration. Three cytokines (GM-CSF, IL-17F, and IL-3) were excluded from further analysis because >80% of their measurements fell outside of their respective standard curves. Forty-four cytokines were thus used for downstream analysis. Individual cytokine measurements that did not have either a) bead counts ≥35 and technical CV≤30%, or b) bead counts ≥20 and technical CV≤15%, were also excluded from analysis.

### Differential Analysis

To better isolate the relationship between molecular features and progression, a number of covariates are adjusted for in our models unless otherwise stated. These covariates include clinical and demographic variables, assay-specific variables, and a variable to adjust for the study from which a biospecimen was collected. Clinical and demographic variables were individually assessed using logistic regression to determine each variable's unadjusted association with progression. All variables that met the criterion of p≤0.20 were considered for inclusion in the “full model”—a multivariable logistic regression with progression as the outcome variable. Beginning with the full model, variables were removed one at a time, in order of descending p-value, and only for variables that have p>0.10. Age and sex were considered key covariates and were thus never removed from the model. The full model also included race, body mass index, and indicators for the following comorbid conditions: cardiovascular and cerebrovascular, metabolic and/or diabetic, renal, and respiratory. After variable selection, the clinical covariates retained in the final model included age, sex, race, and an indicator for comorbid respiratory conditions. Association analyses with Immune Profiler data additionally adjusted for cell viability, neutrophil frequency (as a measure of neutrophil contamination during PBMC isolation), and immune subset recovery count, as these variables were associated with sample quality. Lastly, principal component analysis of the Immune Profiler data showed a batch effect associated with the study from which biospecimens were collected. Therefore, an indicator variable was included to adjust for study.

Univariate differential analysis to identify molecular features differentiating Progressors from Non-progressors was performed using linear modeling methods. For these analyses, each molecular feature was regressed on the outcome group and appropriate clinical and technical covariates. For count-based data such as RNA-, ATAC-, and TaPE-seq, the voom-limma method was used. The method normalizes data using the default “TMM” method in the edgeR package, estimates the mean-variance relationship of the log-counts in order to generate sample weights, and conducts statistical inference of the estimate of association with limma's empirical Bayes analysis pipeline. For non-count-based data (e.g., cell subset frequencies and cytokine levels), differential analysis was performed by fitting generalized linear models (GLMs). Where appropriate, data were transformed (e.g., log transformation) prior to fitting the GLMs.

Following linear modeling, the Benjamini-Hochberg procedure was used to correct for multiple hypothesis testing within each molecular data type. For Immune Profiler data, comparisons were done per cell subset, and the resulting p-values across all tests within a cell subset were corrected for. Significance was assessed at FDR≤0.05; occasionally, differential features were detected at FDR≤0.1.

### Pathway Analysis

Gene sets from MSigDB were used for pathway analysis, and three independent methods were employed. First, enrichment of gene sets for significant differential genes were tested using hypergeometric tests. Second, gene set enrichment analysis (GSEA) was performed using effect estimates from univariate differential analysis, and enabled identification of gene sets where the individual genes may not be significantly differentially expressed but are nevertheless coordinated in their association to progression status. And third, pathway analysis using gene set variation analysis (GSVA) was done by differential analysis of the GSVA pathway enrichment scores.

### TaPE-Seq OligoAb Cocktail

The OligoAb cocktail includes a panel of oligo-barcoded antibodies enabling protein abundance estimation using Targeted Protein Estimation by sequencing (TaPE-seq). The markers targeted by the antibodies in the cocktail are listed in the tables below.

TABLE 7
List of the lineage markers targeted by the antibodies in the cocktail.
Lineage

| | | | |
|---|---|---|---|
| CD3 | CD19 | IL2RA (CD25) | NCAM1 (CD56) |
| CD4 | CR2 (CD21) | IL7R (CD127) | TRDC; TRGC1 |
| | | | (gdTCR) |
| CD8A | FCGR2A/B (CD32) | ITGAX (CD11c) | |
| CD14 | FCGR3A/B (CD16) | MME (CD10) | |

TABLE 8
List of the functional markers targeted by the antibodies in the cocktail.
Functional

| | | | |
|---|---|---|---|
| ADGRG1 (GPR56) | CD47 | ICAM1 (CD54) | LILRB2 (CD85d) |
| ANPEP (CD13) | CD5 | ICOS (CD278) | LY9 (CD229) |
| B3GAT1 (CD57) | CD6 | ICOSLG (CD275) | CD60a (GD3) |
| BTLA (CD272) | CD69 | IL21R (CD360) | NCR1 (CD335) |
| CCR10 | CD7 | IL2RB (CD122) | NCR2 (CD336) |
| CCR2 (CD192) | CD70 | IL4R (CD124) | NTSE (CD73) |
| CCR4 (CD194) | CD80 (B7-1) | IL6R (CD126) | PDCD1 (PD-1) |
| CCR5 (CD195) | CD81 | ITGA1 (CD49A) | PDCD1LG2 (PD-L2) |
| CCR6 (CD196) | CD84 (SLAMF5) | ITGA2 (CD49B) | PECAM1 (CD31) |
| CCR7 (CD197) | CD86 (B7-2) | ITGA4 (CD49D) | PTGDR2 (CD294) |
| CD160 | CD9 | ITGA5 (CD49e) | PTPRC (CD45RA) |
| CD1A | CTLA4 (CD152) | ITGAE (CD103) | PTPRC (CD45RO) |
| CD1C | CX3CR1 (CD183) | ITGAL (CD11a) | PVR (CD155) |
| CD2 | CXCR2 (CD182) | ITGAM (CD11b) | SELL (CD62L) |
| CD200 | CXCR3 (CD183) | ITGB1 (CD29) | SIGLEC7 (CD328) |
| CD226 | CXCR4 (CD184) | ITGB2 (CD18) | SIGLEC9 (CD329) |
| CD244 (2B4) | CXCR5 (CD185) | KLRB1 (CD161) | SLAMF7 (CD319) |
| CD27 | DPP4 (CD26) | KLRD1 (CD94) | SLC3A2; SLC7A5 (CD98) |
| CD274 (PD-L1) | ENTPD1 (CD39) | KLRG1 | TIGIT |
| CD276 (B7-H3) | FASLG (CD178) | KLRK1 (NKG2D) | TNFRSF14 (CD270) |
| CD28 | FCRL3 (CD307c) | LICAM (CD171) | TNFRSF18 (GITR) |
| CD38 | FCRL6 | LAG3 (CD223) | TNFRSF4 (OX40) |
| CD40 | HAVCR2 (TIM-3) | LAIR1 (CD305) | TNFRSF8 (CD30) |
| CD40LG (CD154) | HLA-ABC | LGALS3 (Galectin3) | TNFRSF9 (41BB) |
| CD44 | HLA-DRA | LILRB1 (CD85j) | VTCN1 (B7-H4) |

### Multi-Omic Machine Learning Modeling

Block sparse partial least squares discriminant analysis (block.sPLS-DA) was used in identifying correlated feature scores between RNA-seq and ATAC-seq data that are associated with progression status. Nested cross-validation (CV) was used, where the outer CV is a leave-one-out (LOOCV) and the inner CV performs a 5-fold grid search to identify the best hyperparameters using the validation data's area under the ROC curve (AUC). The hyperparameters used in the grid search were keepX and the design matrix covariance weighting between RNA and ATAC blocks. The top subsets were selected using the outer LOOCV AUC and top features were taken from a model retrained on all data using the optimal hyperparameters.

### Expression Quantitative Trait Loci

To link genes to genome-wide association study (GWAS) risk variants, significant expression quantitative trait loci (eQTLs) were identified.

cis-eQTLs were generated using Matrix eQTL using multiple covariates, including sex, probabilistic estimation of expression residuals (PEER) factors derived from gene expression, and genotype principal components. Analysis was limited to variants within 250 kb of genes. eGenes were considered significant at FDR≤0.1, with this FDR being corrected after filtering eQTLs for genes that were DEGs for progression outcome at significance FDR≤0.1.

### Genetic Associations

The association between genetic variants and progression was tested by regressing progression outcome on genotype, adjusting for age, sex, race, an indicator for comorbid respiratory conditions, and 10 principal components derived from whole genome sequencing data.

### Example 2: Molecular Data Generation

Our aim was to capture the earliest immunological signature upon confirmed SARS-CoV-2 infection paired with clinical COVID-19 outcomes of significant progression for each participant. A total of 162 CPR participants enrolled had evaluable demographic and clinical characteristics (Table 9). Additional participant characteristics included body mass index (BMI), smoking frequency and years of use, and comorbidities classified by affected physiologic systems (Table 10). Upon analyzing these demographics and clinical characteristics, advanced age, female gender, Black race, high BMI, and presence of comorbidities including cardiovascular, metabolic, renal, and respiratory conditions were found to be significant predictors of progression (Table 11). These observations are consistent with previously identified and reported associations with severe COVID-19.

TABLE 9
Demographics and clinical characteristics of participants enrolled in CPR

| | Total | | Progressor | | Non-progressor | |
|---|---|---|---|---|---|---|
| Characteristics | (N = 162) | | (N = 24) | | (N = 138) | |
| Age, mean (SD) | 41.4 | (14.8) | 53 | (18) | 39 | (13) |
| Age category, N (%) | | | | | | |
| 18-29 | 41 | (25.3%) | 2 | (8.3%) | 39 | (28.3%) |
| 20-49 | 75 | (46.3%) | 10 | (41.7%) | 65 | (47.1%) |
| 50-64 | 35 | (21.6%) | 6 | (25%) | 29 | (21%) |
| 65+ | 11 | (6.8%) | 6 | (25%) | 5 | (3.6%) |
| Female, N (%) | 98 | (60.5%) | 20 | (83.3%) | 78 | (56.5%) |
| Race*, N (%) | | | | | | |
| White | 87 | (62.1%) | 8 | (36.4%) | 79 | (66.9%) |
| Black | 45 | (32.1%) | 13 | (59.1%) | 32 | (27.1%) |
| Asian | 7 | (5%) | 0 | (0%) | 7 | (5.9%) |
| American Indian/Alaskan Native | 2 | (1.4%) | 1 | (4.5%) | 1 | (0.8%) |
| Native Hawaiian/Pacific Islander | 1 | (0.7%) | 0 | (0%) | 1 | (0.8%) |
| Race unk/not reported, N (%) | 22 | (13.6%) | 2 | (8.3%) | 20 | (14.5%) |
| Hispanic*, N (%) | 39 | (24.4%) | 5 | (20.8%) | 34 | (25%) |
| Ethnicity unk/not reported, N (%) | 2 | (1.2%) | 0 | (0%) | 2 | (1.4%) |

TABLE 10
Additional clinical characteristics of participants enrolled in CPR

| | Total | | Progressor | | Non-progressor | |
|---|---|---|---|---|---|---|
| Characteristics | (N = 162) | | (N = 24) | | (N = 138) | |
| BMI, mean (SD) | 29.6 | (7.4) | 32 | (7) | 29 | (7) |
| BMI category*, N (%) | | | | | | |
| Underweight | 1 | (0.6%) | 0 | (0%) | 1 | (0.7%) |
| Healthy | 43 | (27.2%) | 5 | (20.8%) | 38 | (28.4%) |
| Overweight | 54 | (34.2%) | 7 | (29.2%) | 47 | (35.1%) |
| Obese | 45 | (28.5%) | 7 | (29.2%) | 38 | (28.4%) |
| Severely obese | 15 | (9.5%) | 5 | (20.8%) | 10 | (7.5%) |
| BMI missing, N (%) | 4 | (2.5%) | 0 | (0%) | 4 | (3%) |
| Current smoker, N (%) | 42 | (25.9%) | 7 | (29.2%) | 35 | (25.4%) |
| Smoking frequency^{#}, N (%) | | | | | | |
| Light use (<1-5 times per day) | 11 | (26.2%) | 1 | (14.3%) | 10 | (28.6%) |
| Moderate use (6-10 times per day) | 10 | (23.8%) | 1 | (14.3%) | 9 | (25.7%) |
| Heavy use (11+ times per day) | 2 | (4.8%) | 0 | (0%) | 2 | (5.7%) |
| Missing | 19 | (45.2%) | 5 | (71.4%) | 14 | (40%) |
| Years smoked^{#}, N (%) | | | | | | |
| <5 years | 9 | (21.4%) | 1 | (14.3%) | 8 | (22.9%) |
| 5-15 years | 7 | (16.7%) | 0 | (0%) | 7 | (20%) |
| 15+ years | 17 | (40.5%) | 5 | (71.4%) | 12 | (34.3%) |
| Missing | 9 | (21.4%) | 1 | (14.3%) | 8 | (22.9%) |
| Comorbid conditions, N (%) | | | | | | |
| Autoimmune | 1 | (0.6%) | 1 | (4.2%) | 0 | (0%) |
| Cardiovascular/cerebrovascular | 37 | (22.8%) | 12 | (50%) | 25 | (18.1%) |
| Hematologic | 6 | (3.7%) | 1 | (4.2%) | 5 | (3.6%) |
| Metabolic | 25 | (15.4%) | 8 | (33.3%) | 17 | (12.3%) |
| Neurologic | 4 | (2.5%) | 1 | (4.2%) | 3 | (2.2%) |
| Renal | 7 | (4.3%) | 3 | (12.5%) | 4 | (2.9%) |
| Respiratory | 16 | (9.9%) | 5 | (20.8%) | 11 | (8%) |
| Other | 49 | (30.2%) | 8 | (33.3%) | 41 | (29.7%) |

*Percentages calculated out of partiopants with non-missing dats
^{#}Percentages calculated out of current smokers

TABLE 11
Statistical demographic and characteristic predictors of progression

| Characteristic | Category | Coefficient | p-value |
|---|---|---|---|
| Age (continuous) | per year | 0.059514 | <0.001 |
| Age (categorical) | 18-29 (ref) | — | — |
| | 30-49 | 1.098612 | 0.17 |
| | 50-64 | 1.394878 | 0.10 |
| | 65+ | 3.152736 | <0.001 |
| Age (binary) | 65+ vs. not | 2.182299 | <0.001 |
| Sex | Female (vs. male) | 1.347074 | <0.05 |
| Race | White (vs. not) | −0.985058 | <0.05 |
| | Black (vs. not) | 1.364757 | <0.01 |
| Ethnicity | Hispanic (vs. not) | −0.236389 | 0.66 |
| Smoking | Current (vs. not) | 0.192078 | 0.69 |
| BMI | per unit | 0.051757 | 0.052 |
| BMI | Under/Healthy (BMI <25) | — | — |
| (categorical) | (ref) | | |
| | Overweight (BMI 25-<30) | 0.149886 | 0.81 |
| | Obese (BMI 30-<40) | 0.362448 | 0.56 |
| | Severely Obese (BMI 40+) | 1.360977 | 0.060 |
| BMI (binary) | Severely Obese (vs. not) | 1.182695 | <0.05 |
| Comorbidities | Autoimmune (vs. not) | 22.357828 | 1.00 |
| | Cardio/Cerebrovascular | 1.508512 | <0.01 |
| | (vs. not) | | |
| | Hematologic (vs. not) | 0.145417 | 0.90 |
| | Metabolic (vs. not) | 1.269430 | <0.05 |
| | Metabolic, Diabetes (vs. not) | 1.756041 | <0.01 |
| | Metabolic, Other (vs. not) | 0.928363 | 0.11 |
| | Neurologic (vs. not) | 0.671168 | 0.57 |
| | Renal (vs. not) | 1.565635 | 0.050 |
| | Respiratory (vs. not) | 1.111291 | 0.061 |
| | Other (vs. not) | 0.167992 | 0.72 |

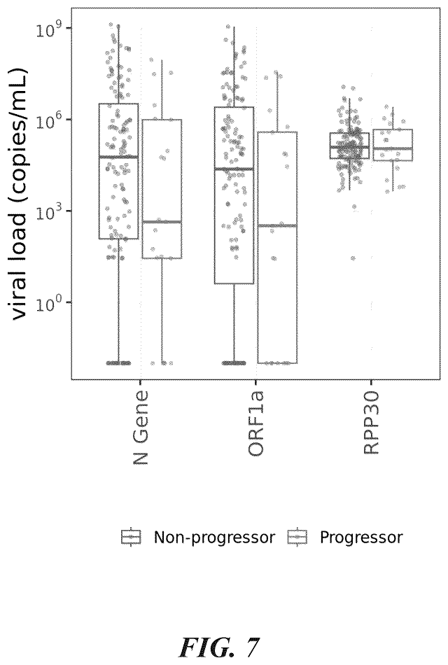

Participant blood samples and other biospecimens had an overall mean time to collection of 3.2 days post-positive test. Peripheral blood from 158 participants was processed through a multi-omic immunogenomic profiling workflow, consisting of FACS immune cell subset sorting followed by molecular sequencing-based assays for RNA-seq, ATAC-seq, TaPE-seq, and WGS. The 24 immune cell subsets resolved by fluorescent surface marker staining and sorting represent canonically defined subsets with respect to the ability to immunophenotype by cell surface markers, and known characteristics of lineage and functional differentiation (FIG. 6). An additional sorted bulk PBMC subset comprised the 25th subset, but was used as a quality control metric and was not further analyzed. Molecular features generated for each participant PBMC sample were analyzed along with measures of viral load from mid-turbinate swabs and plasma cytokine levels (Table 12). No significant differences in the viral load early in disease course were detected, as measured by detection of the viral genes N and ORF1a by ddPCR or in the IgG antibody reactivity towards SARS-CoV-2 S and N protein or other related human coronaviruses (FIG. 7).

TABLE 12
Molecular features generated from each participant

| Molecular assays | Features measured |
|---|---|
| Viral load | 3 |
| Luminex - cytokine panel | 47 |
| Immune Profiler | |
| Cell frequencies | 74 |
| RNA-seq | 60,049 (x24 subsets) |
| ATAC-seq | 260,056 (x24 subsets) |
| TaPE-seq | 117 (x24 subsets) |

### Example 3: Progression is Associated with Early Changes in Monocyte and T Cell Subsets

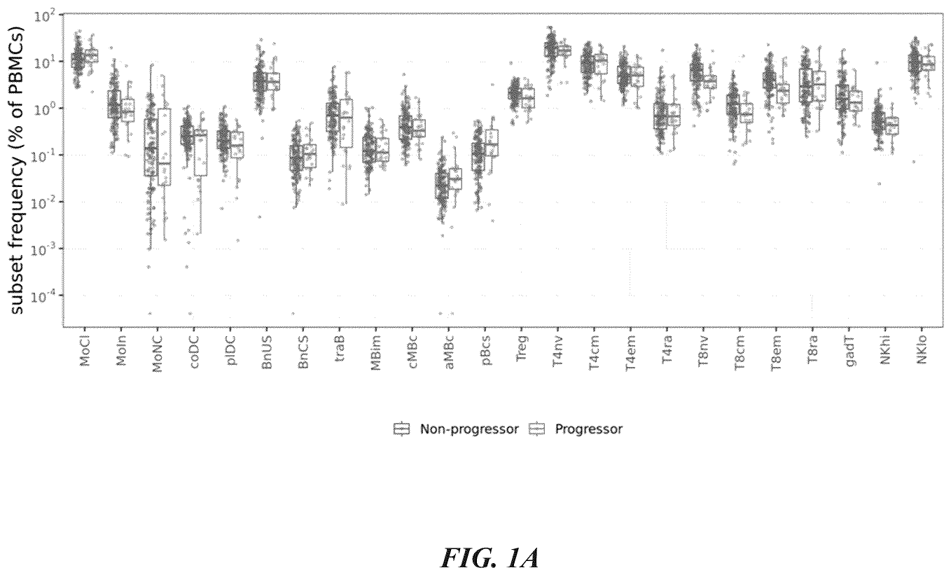

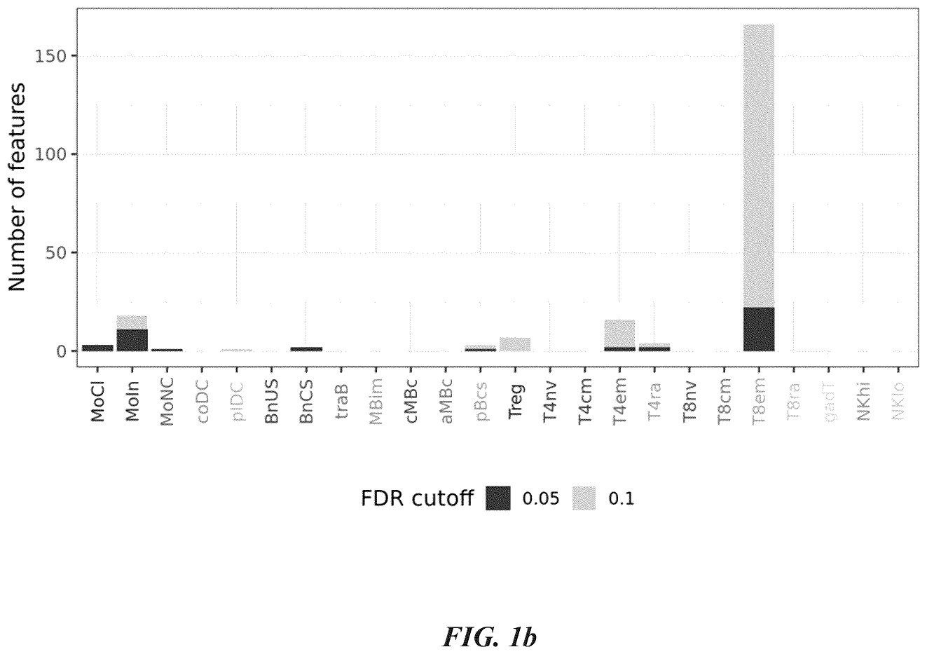

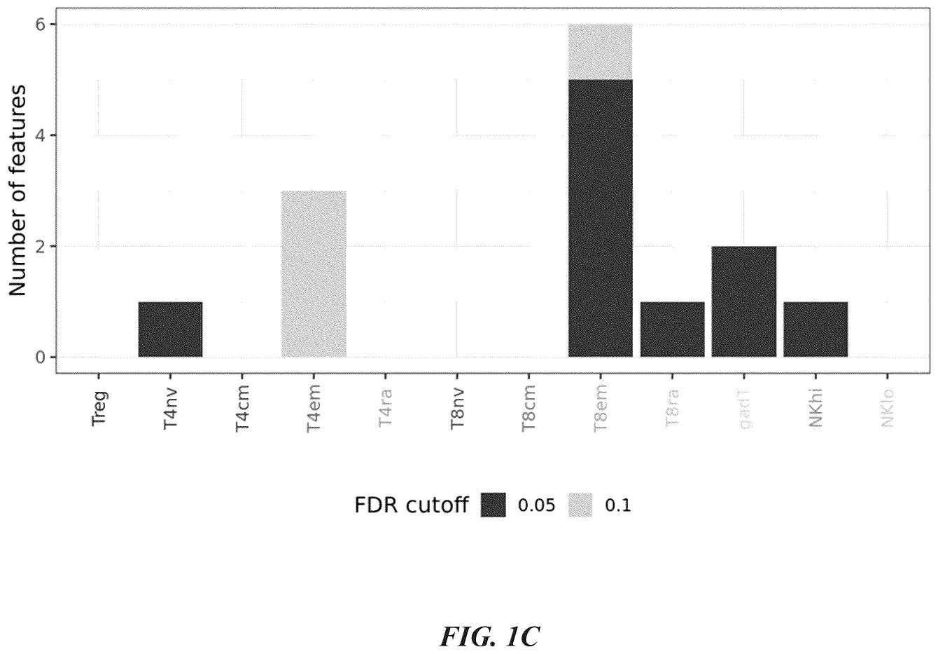

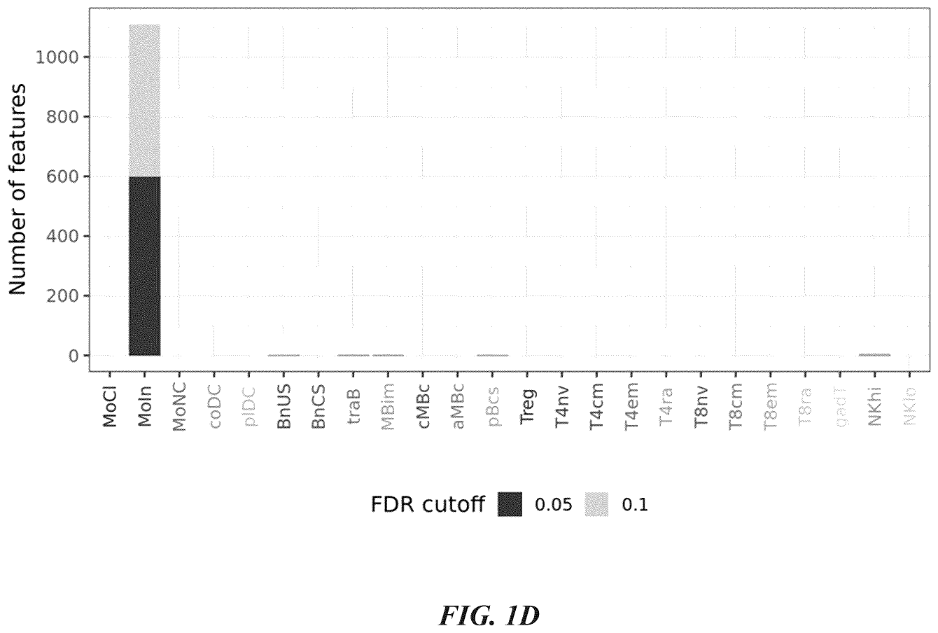

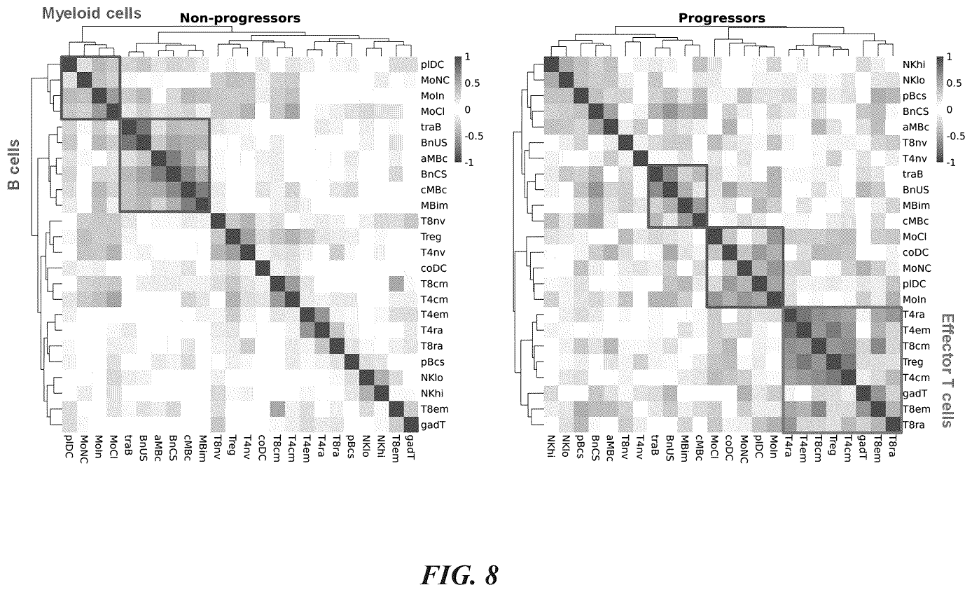

Progressor and Non-progressor cell subset frequencies (as a percentage of PBMCs) across the 24 subsets were assessed, and no significant differences were observed after adjusting for multiple testing (FIG. 1A), suggesting that early timepoints of progression are not correlated with cell frequency shifts when viewed across the global PBMC population. To assess whether higher resolution molecular phenotyping could reveal more nuanced phenotypes, a multi-omic approach that measures over 8 million molecular features per participant was employed (Table 12). Specifically, a series of correlation, differential, and multi-omic analyses were performed to identify immunological differences between Progressors and Non-progressors. A pairwise correlation analysis was conducted to identify immune lineages that may be coordinated in the early immune response to infection (FIG. 8). Progressors and Non-progressors showed some clustering of myeloid and B cell subsets, while an effector T cell cluster was seen only in Progressors.

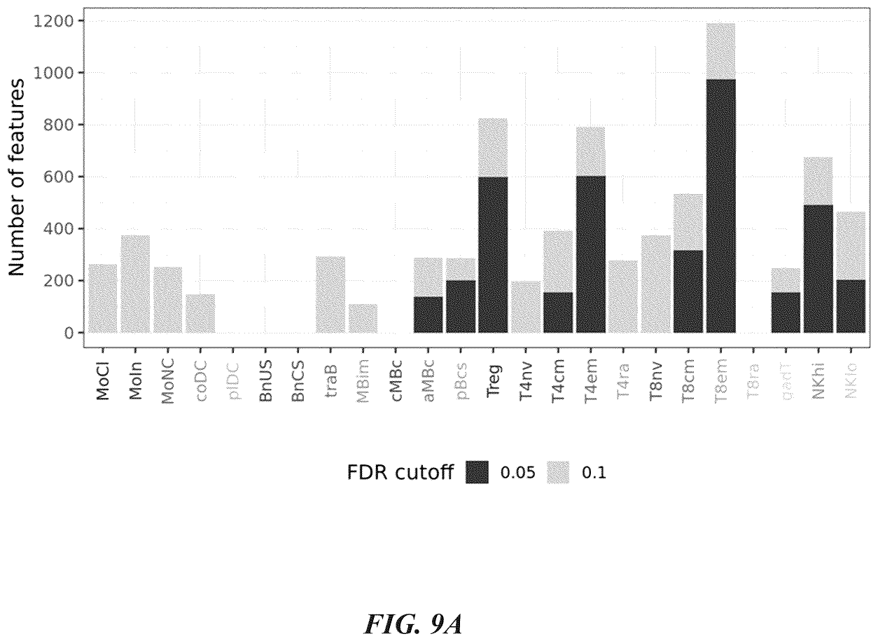

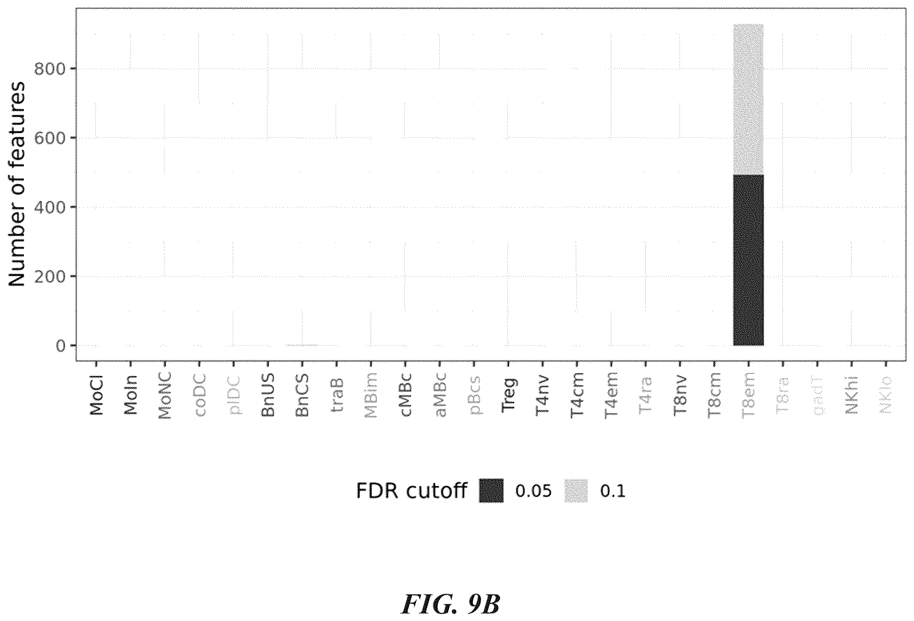

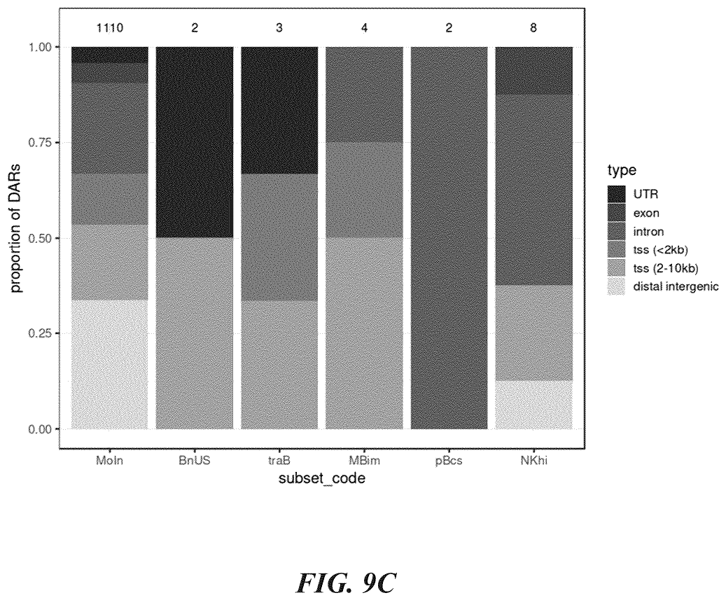

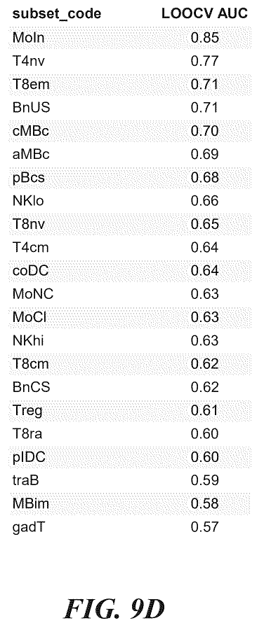

Differential expression analysis identified transcripts in specific cell subsets up- or down-regulated in Progressors (FIG. 1B). The highest number of differentially expressed genes (DEGs) was seen in the CD8+ effector memory (T8em) cell subset, followed by the intermediate monocytes (MoIn; CD14+CD16+) subset. Pathway analysis using Gene Set Enrichment Analysis (GSEA) and Gene Set Variation Analysis (GSVA) showed the highest number of significant pathways in the T8em cell subset (FIGS. 9A and 9B). Assessment of cell surface protein expression on the T cell and NK cell subsets revealed the highest number of differentially expressed proteins (DEPs) in the T8em subset (FIG. 1C). The majority of differentially accessible regions (DARs) identified with ATAC-seq were observed in the MoIn subset (FIGS. 1D, 9C, and Table 4).

Multi-omic approaches that integrate “regulatory layer” evidence from functional genomic or genetic assays can build confidence in DEGs identified from differential expression analysis by linking them to genetic factors. To this end, an approach to identify expression quantitative trait loci (eQTLs) was employed that correspond to the DEGs identified from differential analysis. Cell subset-resolved cis-eQTLs were employed, and DEGs (at FDR≤0.1) in T8em and MoIn were also subset-resolved cis-eQTLs (Table 13, Table 3, and Table 5).

TABLE 13
DEGs detected in each cell subtype (FDR ≤0.1)

| Subset | Gene names |
|---|---|
| MoIn | C1QB, MCEMP1, S100A9, THBD, TRPM2 |
| MoNC | LAIR2 |
| T4em | ACAD9, TK1 |
| T4ra | DFNB31 |
| T8em | ACAA2, BIRC5, CD53, CD59, CISD1, COL6A2, FDXR, |
| | GTF2IRD2, HADH, HDHD3, IFI27, ISOC2, MRPL27, |
| | MYO15B, NFIA, OTOF, PRDX1, RPL26L1, SESTD1, |
| | TIMM13, TXNDC17 |

Finally, multi-omic machine learning was used to identify cell subsets with the most discriminative features for classifying progression outcome. Block sparse partial least squares-discriminant analysis was performed for each cell subset utilizing gene expression, chromatin accessibility, and progression outcomes. This analysis showed MoIn, naive CD4+ T cells, and T8em as the top three cell subsets whose RNA and ATAC features best classified progression outcome (FIG. 9D).

In summary, this high-resolution multi-omic approach revealed significant immune signatures already differentiating Progressors from Non-progressors during early infection before clinical intervention. Understanding what cell subsets had the greatest molecular divergence between the two outcome groups helped guide further cell subset-specific analysis, with a focus on monocytes and T8em cells.

### Example 4: Early Inflammatory Cytokine and Chemokine Responses in Progressors

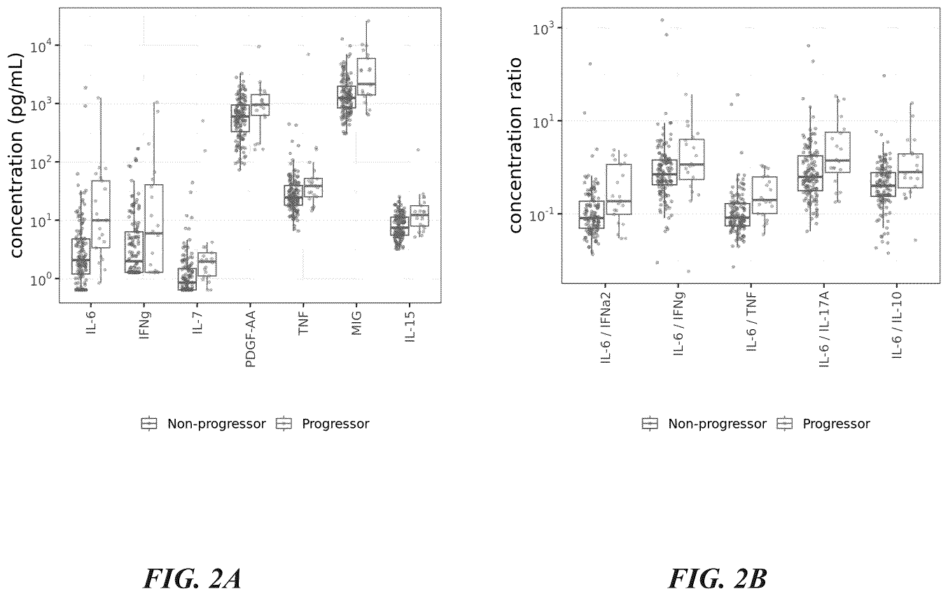

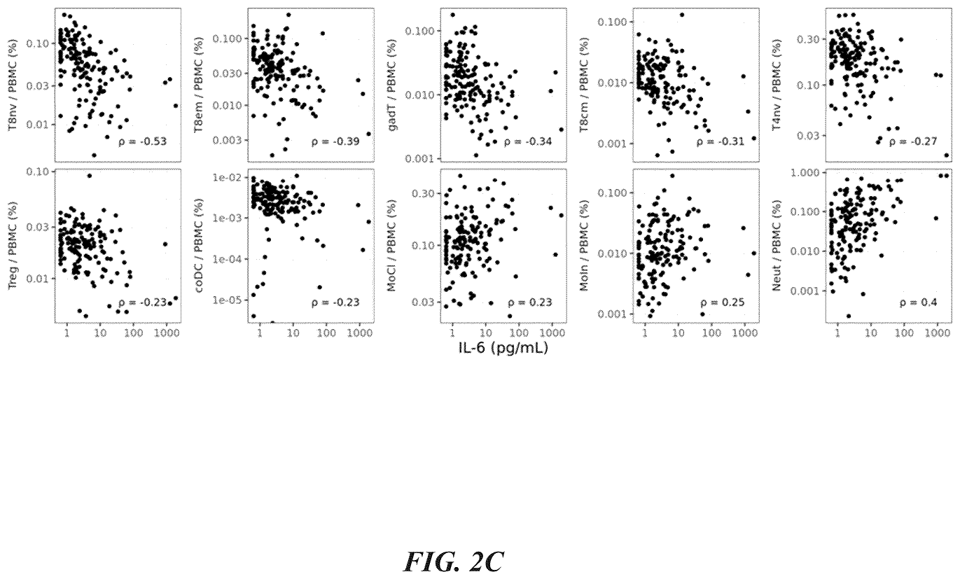

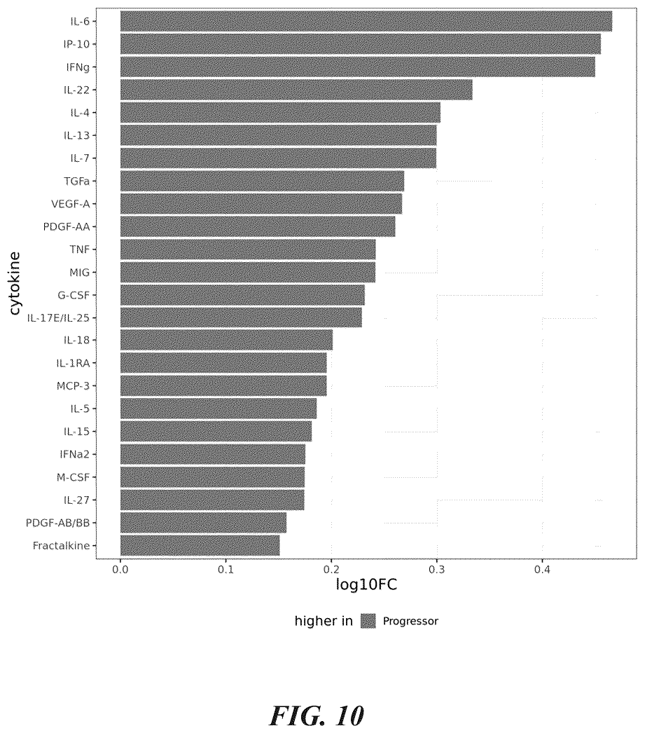

To determine the association between plasma cytokines and COVID-19 disease progression, 44 total cytokines, chemokines, and growth factors were analyzed in plasma during this early phase of SARS-CoV-2 infection. At an FDR≤0.05, seven cytokines (IL-6, IFNγ, IL-7, PDGF-AA, TNF, MIG, and IL-15) were significantly higher in Progressors (FIG. 2A, Table 1). At an FDR≤0.1, 24 total cytokines were higher in Progressors (FIG. 10), including key inflammatory cytokines such as IL-6, IP-10, IFNγ, TNF, and IL-18, as well as chemokines such as MCP-3 (CCL7), MIG (CXCL9), IP-10 (CXCL10), and Fractalkine (CX3CL1) (FIG. 10).

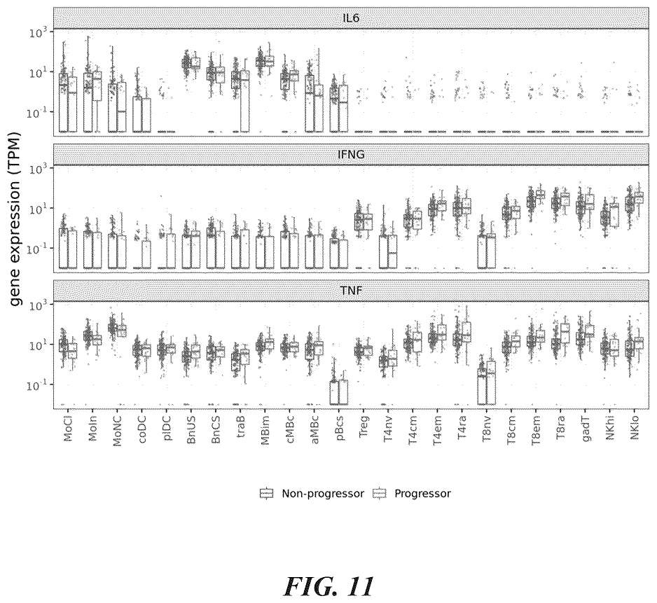

IL-6 was the cytokine with the largest difference in magnitude between Progressors and Non-progressors (FIGS. 2A and 10). As cytokines are often produced in a cascade, various cytokine ratios were evaluated to help discern the type of immune response and immune regulation occurring in COVID-19 progression. Ratios of IL-6 and common type I interferons (e.g., IFNα2) and Th1 (e.g., IFNγ and TNF), Th17 (e.g., IL-17A) and anti-inflammatory (e.g., IL-10) cytokines were computed within individuals. For each cytokine ratio, the IL-6 response relative to the other common cytokines trended higher in Progressors compared to Non-progressors (FIG. 2B). Cytokine gene expression levels in myeloid, B, and T cell subsets were also evaluated by RNA-seq to better disentangle the various contributions by cells to cytokine production (FIG. 11). Focusing on the key inflammatory cytokine genes IL6, IFNγ, and TNF, expression of IL6 was found to trended higher in intermediate monocytes in Progressors, whereas expression of IFNγ and TNF in Progressors trended higher in the CD4+ and CD8+ T cell subsets (FIG. 11).

Plasma concentrations of IL-6, and to a lesser extent IL-10, correlate with increases in CD14+ monocytes with phenotypic similarity to myeloid-derived suppressor cells, which are capable of suppressing T cell proliferation. Plasma IL-6 concentrations was found to be negatively correlated with the frequencies of a number of circulating T cell subsets, including subsets of conventional CD4+ and CD8+ T cells, regulatory T cells, and gamma delta T cells, and positively correlated with the frequencies of classical (MoCl; CD14+CD16−) and MoIn monocytes and neutrophils (FIG. 2C).

### Example 5: Cellular and Molecular Perturbations in the Monocyte Compartment of Progressors

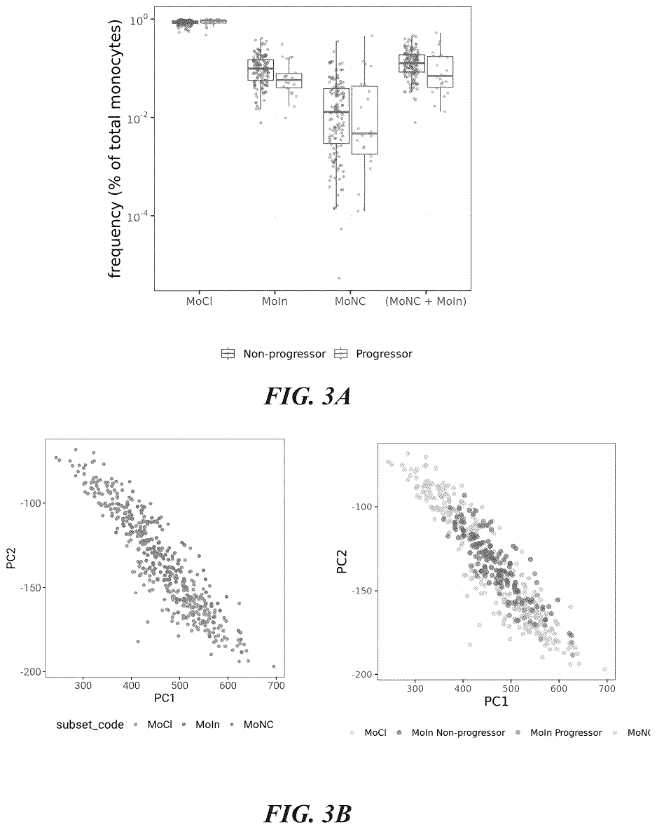

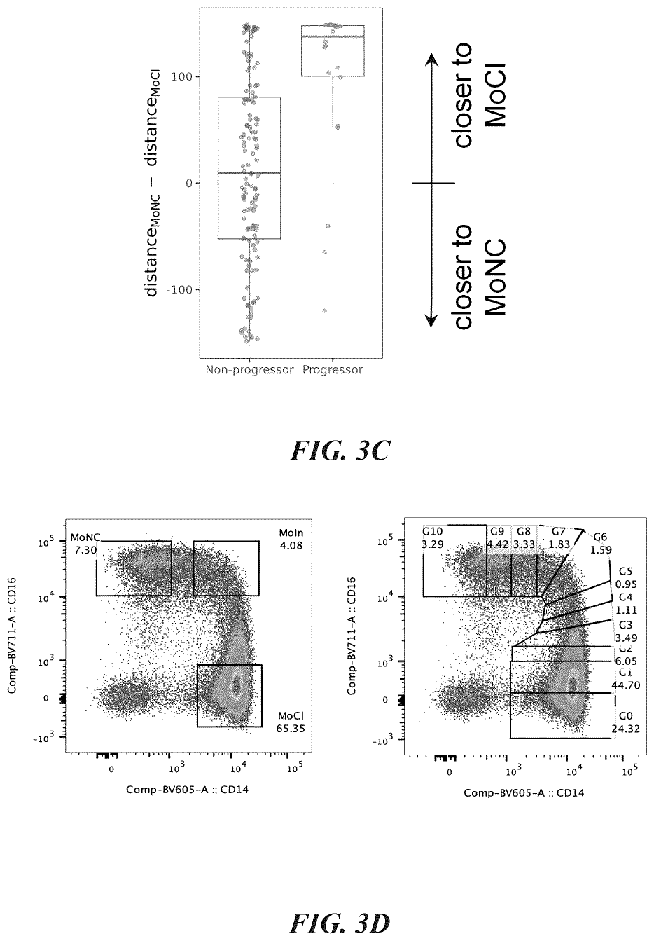

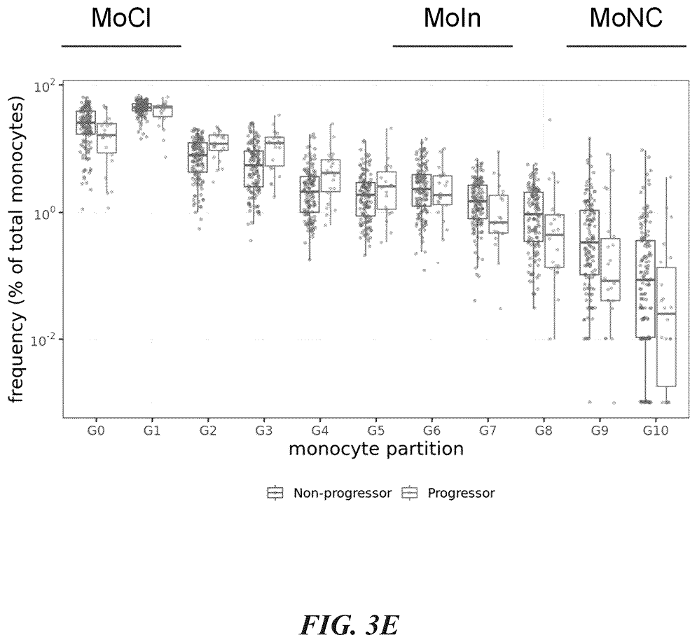

Relative frequencies measured by cytometry in the total monocyte population were investigated and the ratios of MoIn, MoNC, and CD16+ (MoIn+MoNC) over the total monocytes were found to all be significantly lower in Progressors (p≤0.01, p≤0.05, p≤0.001; respectively; FIG. 3A, Table 2). By contrast, the ratio of MoCl over total monocytes was not significantly different between the two groups. Measuring chromatin accessibility of each subset further revealed differences in monocyte populations between the two outcome groups. Principal component analysis (PCA) of accessible chromatin regions in the three monocyte subsets recapitulated the known developmental trajectory from MoCl, to MoIn, to MoNC, and showed a trend where Progressor MoIn grouped more closely with MoCl (FIG. 3B). By determining the distances of Progressor and Non-progressor MoIn from the MoCl and MoNC centroids in principal component space, the MoIn of Progressors were observed to be clustered significantly more closely to MoCl and farther from MoNC (FIG. 3C).

To further investigate the observed differences in monocyte development and identity, a finer gating method was adapted for distinguishing monocyte subsets by flow cytometry using the cell surface markers CD14 and CD16. Partitioning monocytes by CD14 and CD16 positions cells along a developmental path from CD14+CD16− MoCl, through CD14+CD16+ MoIn, to CD14-CD16+ MoNC (FIG. 3D). The frequencies of each gated partition showed a trend of increasing frequencies for Progressors as the gating moved from the MoCl to MoIn partitions (G2 to G5, FIG. 3E). This trend reversed upon reaching the CD14+CD16+ MoIn region with Progressors showing decreasing cell frequencies compared to Non-progressors in the partitions gated between MoIn and MoNC (G6 to G10, FIG. 3E). These findings are consistent with the model that circulating monocytes egress to sites of pathology, preventing the peripheral development of more mature MoIn and MoNC subsets.

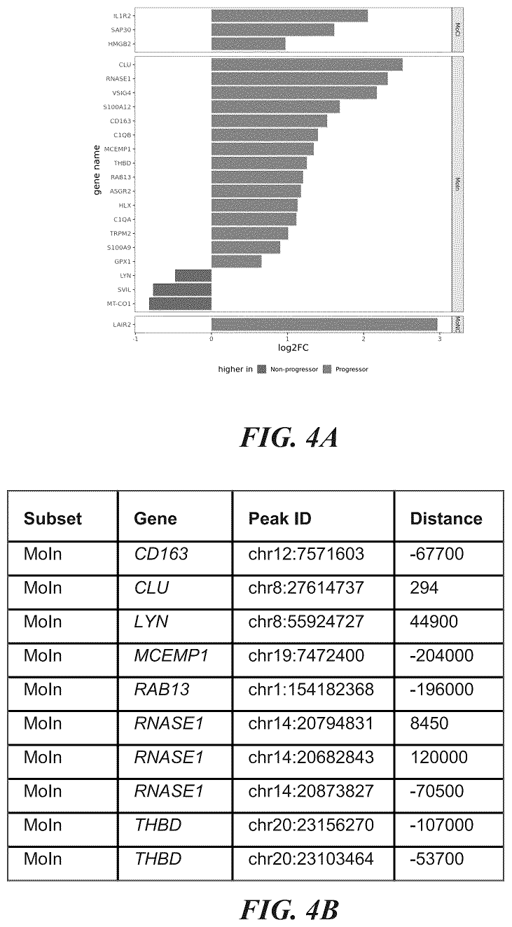

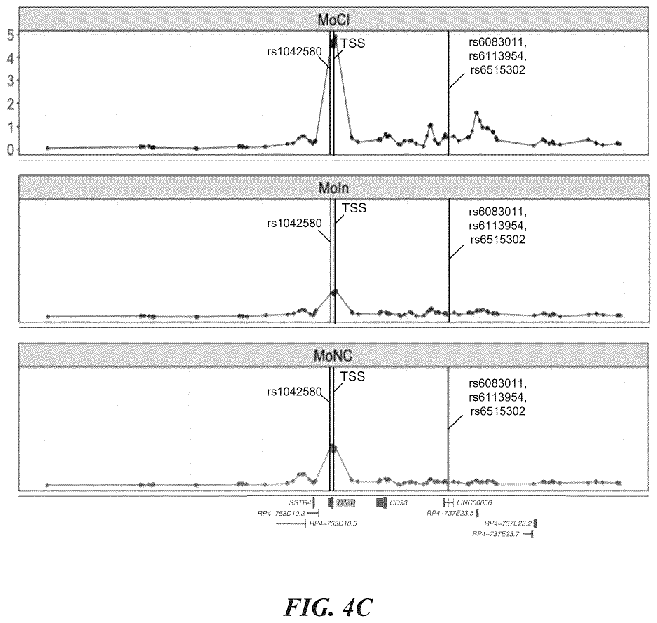

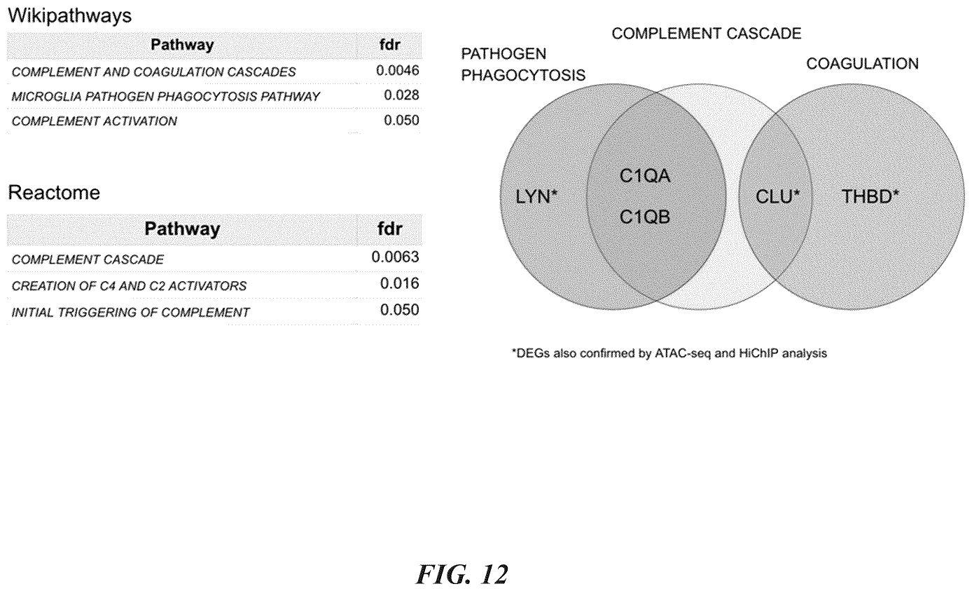

The DEGs and DARs in monocytes further revealed differences between Progressors and Non-progressors. Monocytes of Progressors showed increased expression in genes and pathways that have been previously associated with later stage severe COVID-19 (FIGS. 4A, 12A, and 12B). At FDR≤0.1, CD163, CLU, LYN, MCEMP1, RAB13, RNASE1, and THBD were found to be differentially expressed between the two groups and were also proximal to DARs (FIG. 4B). CD163 expression in monocytes and soluble CD163 in the serum is correlated with disease, and MCEMP1 and THBD have been identified in other studies as having prognostic value for severe COVID-19. Interestingly, these two genes also had significant (FDR≤0.1) cis-eQTLs (Table 13), indicating that there may be upstream genetic and epigenetic factors contributing to their differential expression in monocytes.

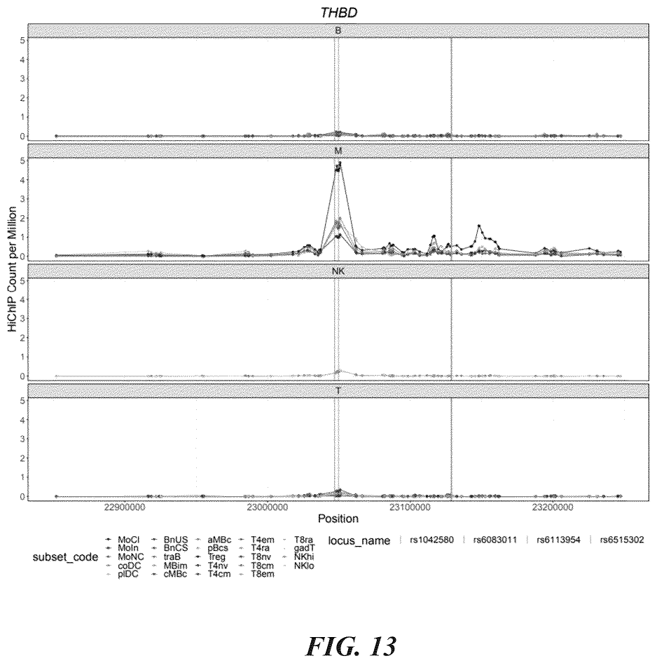

A genetic variant in THBD (rs1042580) is associated with risk of ICU admission and mortality from COVID-19. This variant and the significant variants identified as eQTLs (rs6083011, rs6113954, rs6515302) were not independently associated with progression status in our study (unadjusted p>0.4 for all four SNPs). However, chromatin contact mapping in healthy donors indicated that the 3 eQTL SNPs fall in regions with evidence of physical contact with the THBD promoter region in MoCl, but not MoIn (FIGS. 4C and 13). In contrast, rs1042580 is in the 3′UTR. Taken together, these results suggest that THBD may influence progression and identify two candidate regulatory mechanisms for THBD: 1) genetic variation in the three eQTL SNPs influencing chromatin contact and subsequent gene expression, and 2) genetic variation in the 3′UTR influencing another regulatory mechanism such as mRNA localization, stability, or translation.

### Example 6: Effector Memory CD8+ T Cells from Progressors Show Strong Early Activation and Possible Dysfunction

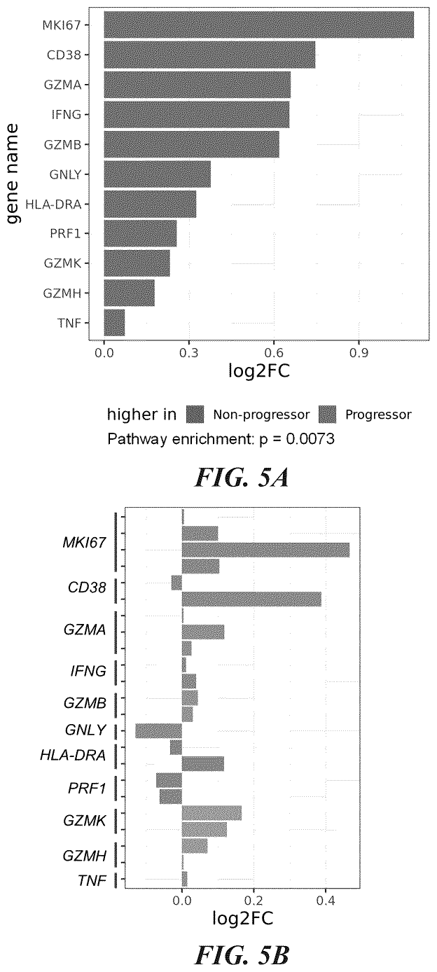

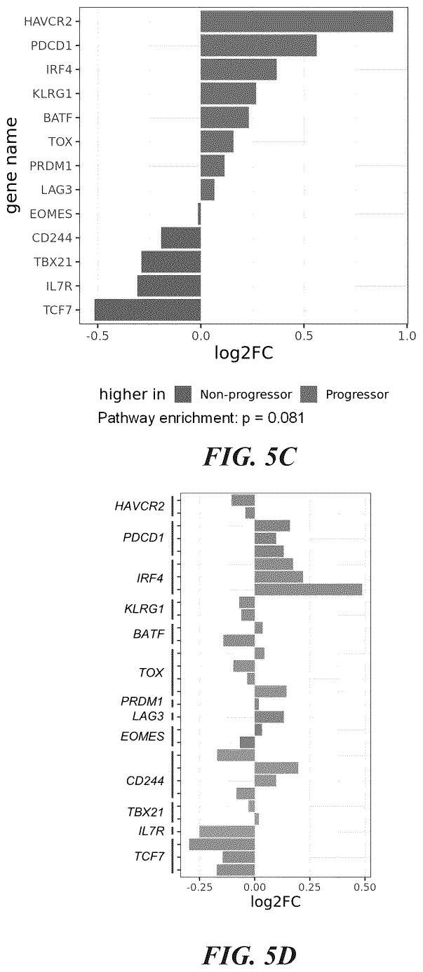

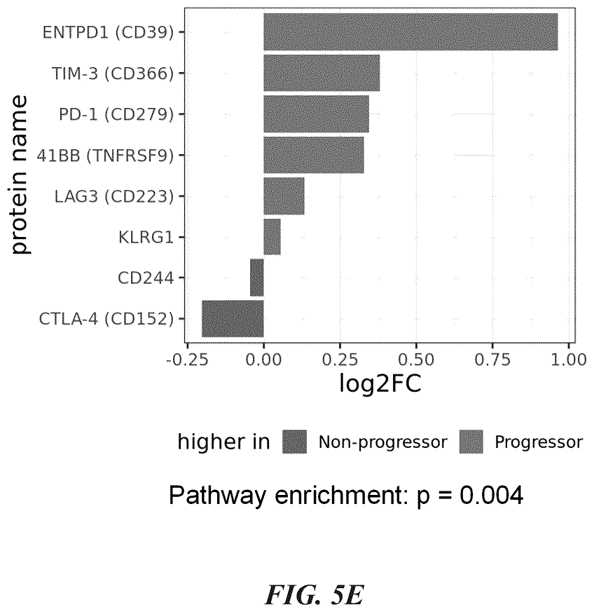

Several studies have linked strong activation in the CD8+ T cell compartment to severe COVID-19. Using GSEA, T8em in Progressors showed a strong enrichment (p=0.0073) of genes involved in an activation-related signature, displaying increased expression of markers of cell proliferation (MKI67), surface markers of activation (CD38, HLA-DRA), cytokines (IFNγ, TNF), and cytolytic factors (GZMA, GZMB, GNLY, PRF1, GZMK, GZMH) (FIG. 5A). These changes were also apparent at the chromatin level, as Progressor T8em showed increased chromatin accessibility around the cell proliferation marker Ki67 (MKI67) and granzyme (GZMK, GZMH, GZMB, GZMA) chromatin regions, as well as increased accessibility around activation-related regions (CD38, HLA-DRA) (FIG. 5B). In particular, granzyme A (GZMA) was increased in Progressors by differential analysis at FDR≤0.1. These findings suggest that Progressors exhibit a strong activation signature in the T8em compartment even at this early phase of infection.

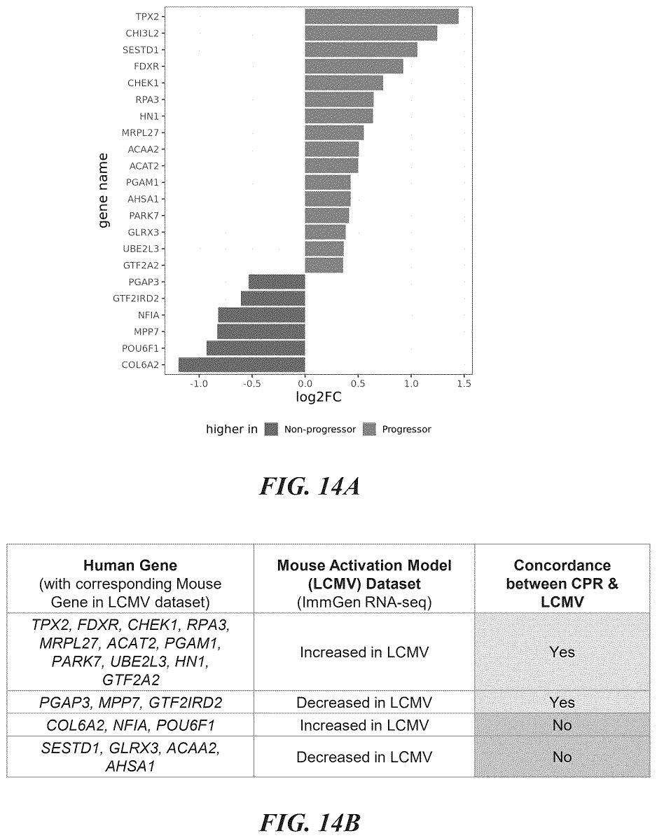

The T8em subset also showed the highest number of DEGs (FIG. 2B). To better understand the function of these DEGs, the 22 significant genes in Progressors (FDR≤0.05) (FIG. 14A) were compared with DEGs from a LCMV murine model of viral-induced T cell activation. Twenty-one of these DEGS in Progressor T8em were differentially regulated in activated CD8+ effector T cells from LCMV-infected mouse spleens relative to naive splenic CD8+ T cells (FIG. 14B). Fourteen of the 21 genes matched the directionality of expression in the mouse model of T cell activation and the Progressor outcome group (i.e., increased in both Progressors and in the LCMV murine model, or decreased in both), suggesting that T8em cells from Progressors may be enriched for genes involved in virally-induced T cell activation.

Several studies have found indications of both effector- and dysfunction-related signatures in the CD8+ T cell compartment of SARS-CoV2-infected individuals. Progressor T8em expressed increased levels of regulatory cell surface markers (HAVCR2, PDCD1, KLRG1, LAG3) and effector-related transcription factors (IRF4, BATF, TOX, PRDM1) while simultaneously showing a decrease in the expression of memory or persistence-related markers (TBX21, IL7R, TCF7) by GSEA (enrichment p=0.081) (FIG. 5C), which could support either an effector or dysfunction-related signature. At the chromatin level, T8em in Progressors showed increased accessibility at the PDCD1 and IRF4 regions while showing concurrent decreased accessibility around the IL7R region (FIG. 5D), providing some evidence of concordance at the chromatin level. Cell surface protein levels in Progressor T8em also showed an increase in the levels of proteins related to regulatory receptors (TIM-3, PD-1, LAG3, KLRG1) and activation markers (CD39) (p=0.004) (FIG. 5E). Altogether, these data support a model of strong CD8+ T cell activation, and possibly dysfunction, consistent with viral infection.

1. A method for determining a signature for identifying a subject at risk of a severe reaction to a SARS-CoV virus comprising:
(i) obtaining a blood sample from a subject who has tested positive for the SARS-CoV virus;
(ii) identifying a cell from the blood sample of the subject, wherein the cell is an intermediate monocyte (Moln), non-classical monocyte (MoNC), classical monocyte (MoCl), and/or CD8+ effector memory T cell; and
(iii) performing an assay on the cell to detect a feature comprising:
(a) a first differentially expressed gene (DEG) in the Moln, MoNC, or MoCl
(b) a second DEG in the CD8+ effector memory T cell, and/or
(c) elevated chromatin accessibility in the Moln;
wherein the signature comprises the feature.

2. The method of claim 1, wherein expression of the first DEG in the subject is different by a predetermined first amount than the expression of the first DEG in a control subject.

3. The method of claim 1, wherein the first DEG comprises a gene associated with coagulation, complement cascade, and/or pathogen phagocytosis.

4. The method of claim 1, wherein the first DEG comprises a gene in a IL1R2, SAP30, and/or HMGB2 pathway in the MoCl.

5. The method of claim 4, wherein the first DEG comprises IL1R2, SAP30, and/or HMGB2 in the MoCl.

6. The method of claim 1, wherein the first DEG comprises a gene in a ASGR2, C1QA, C1QB, CD163, CLU, COX1, GPX1, HLX, LYN, MCEMP1, MT-CO1, RAB13, RNASE1, S100A12, S100A9, SVIL, THBD, TRPM2, and/or VSIG4 pathway in the Moln.

7. The method of claim 6, wherein the first DEG comprises ASGR2, C1QA, C1QB, CD163, CLU, COX1, GPX1, HLX, LYN, MCEMP1, MT-CO1, RAB13, RNASE1, S100A12, S100A9, SVIL, THBD, TRPM2, and/or VSIG4 in the Moln.

8. The method of claim 1, wherein the first DEG comprises a gene in a LAIR2 pathway in the MoNC.

9. The method of claim 8, wherein the first DEG comprises LAIR2 in the MoNC.

10. The method of claim 1, wherein the first DEG is located a predetermined first distance from a differentially accessible region (DAR) or the second DEG is located a predetermined second distance from the differentially accessible region (DAR).

11. The method of claim 10, wherein the DAR is determined by ATAC-seq.

12. The method of claim 1, wherein expression of the second DEG in the subject is different by a predetermined second amount than the expression of the second DEG in a control subject.

13. The method of claim 1, wherein the second DEG comprises a gene associated with T cell activation.

14. The method of claim 1, wherein the second DEG comprises a gene in a ACAA2, ACAT1, ACAT2, ACTB, ADA, AFMID, AHCY, AHSA1, ALOX5, ANKRD23, ANXA2, ANXA5, APOBEC3B, ARPC1A, ASNA1, ATP5C1, ATP5E, ATP6V1F, BIRC5, BOP1, C19orf54, CALM3, CCDC130, CCL3, CCT4, CCT8, CD53, CD59, CD70, CDK4, CDKN3, CENPM, CHEK1, CHI3L2, CIRBP, CISD1, CKS1B, CLASP1, CLIC1, CLTA, CNIH1, COL6A2, COPS5, COX5A, COX6A1, CTSC, DBI, DOCK10, EIF1AD, ENO1, ENTPD4, ERAL1, FAM160B2, FARP2, FDXR, FKBP1A, GALK1, GAPDH, GGT7, GIGYF1, GLRX3, GNG5, GTF2A2, GTF2IRD2, GTF3C6, GZMA, HADH, HAUS1, HDHD3, HIST1H2BD, HMOX1, HN1, HNRNPC, HPRT1, IFI27, ISOC2, LAMTOR1, LMNB2, LYPLA1, MBTPS1, MCM4, MDH1, MECP2, METTL16, MPP7, MPST, MRPL27, MRPL42, MRPS14, MRPS26, MTCH2, MYL6, MYO15B, NAA38, NAT1, NDUFA2, NDUFB3, NDUFB6, NFIA, NHP2, NOP10, NR2C2, NUF2, OSTC, OTOF, PARK7, PGAM1, PGAP3, PLK1, POP4, POU6F1, PPIA, PPIL1, PRDX1, PRDX3, PRELID1, PSMA2, PSMA6, PSMB2, RHOA, RNPC3, RP11-500M8.7, RPA3, RPL26L1, SDF2, SDF2L1, SEC11A, SENP6, SESTD1, SHFM1, SKA2, SLC25A5, SLC29A2, SLC38A6, SLK, SMS, SNRPB, SNRPD3, SORL1, SRSF8, STMN1, STOML2, TAF1, TALDO1, TIMM13, TKT, TMEM155, TNRC6B, TPGS2, TPI1, TPX2, TSC1, TTLL3, TUBGCP6, TXN, TXNDC17, UBE2L3, UCHL3, UQCR10, UQCRH, VRK2, VTA1, ZBTB20, ZNF275, ZNF395, and/or ZNF518A pathway.

15. The method of claim 1, wherein the second DEG comprises ACAA2, ACAT1, ACAT2, ACTB, ADA, AFMID, AHCY, AHSA1, ALOX5, ANKRD23, ANXA2, ANXA5, APOBEC3B, ARPC1A, ASNA1, ATP5C1, ATP5E, ATP6V1F, BIRC5, BOP1, C19orf54, CALM3, CCDC130, CCL3, CCT4, CCT8, CD53, CD59, CD70, CDK4, CDKN3, CENPM, CHEK1, CHI3L2, CIRBP, CISD1, CKS1B, CLASP1, CLIC1, CLTA, CNIH1, COL6A2, COPS5, COX5A, COX6A1, CTSC, DBI, DOCK10, EIF1AD, ENO1, ENTPD4, ERAL1, FAM160B2, FARP2, FDXR, FKBP1A, GALK1, GAPDH, GGT7, GIGYF1, GLRX3, GNG5, GTF2A2, GTF2RD2, GTF3C6, GZMA, HADH, HAUS1, HDHD3, HIST1H2BD, HMOX1, HN1, HNRNPC, HPRT1, IFI27, ISOC2, LAMTOR1, LMNB2, LYPLA1, MBTPS1, MCM4, MDH1, MECP2, METTL16, MPP7, MPST, MRPL27, MRPL42, MRPS14, MRPS26, MTCH2, MYL6, MYO15B, NAA38, NAT1, NDUFA2, NDUFB3, NDUFB6, NFIA, NHP2, NOP10, NR2C2, NUF2, OSTC, OTOF, PARK7, PGAM1, PGAP3, PLK1, POP4, POU6F1, PPIA, PPIL1, PRDX1, PRDX3, PRELID1, PSMA2, PSMA6, PSMB2, RHOA, RNPC3, RP11-500M8.7, RPA3, RPL26L1, SDF2, SDF2L1, SEC11A, SENP6, SESTD1, SHFM1, SKA2, SLC25A5, SLC29A2, SLC38A6, SLK, SMS, SNRPB, SNRPD3, SORL1, SRSF8, STMN1, STOML2, TAF1, TALDO1, TIMM13, TKT, TMEM155, TNRC6B, TPGS2, TPI1, TPX2, TSC1, TTLL3, TUBGCP6, TXN, TXNDC17, UBE2L3, UCHL3, UQCR10, UQCRH, VRK2, VTA1, ZBTB20, ZNF275, ZNF395, and/or ZNF518A.

16. (canceled)

17. (canceled)

18. The method of claim 1, further comprising determining a ratio of the Moln and/or MoNC to total monocytes, wherein the feature further comprises reduced ratio of the Moln and/or MoNC to the total monocytes.

19. The method of claim 18, wherein the reduced ratio of the Moln and/or MoNC to the total monocytes is a subject ratio of Moln to total monocytes that is (1) lower by a predetermined sixth amount than a control ratio of Moln to total monocytes and/or a subject ratio of MoNC to total monocytes that is lower than a control ratio MoNC to total monocytes; or (2) lower than a predetermined Moln ratio and/or a subject ratio of MoNC to total monocytes that is lower than a predetermined MoNC ratio.

20. (canceled)

21. The method of claim 1, further comprising measuring a cytokine from serum from the blood sample, wherein the feature further comprises:
(i) elevated serum concentration of a first cytokine comprising Fractalkine, G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα, and/or VEGF-A; or
(ii) elevated ratio of IL-6 to a second cytokine comprising Fractalkine, G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα, and/or VEGF-A.

22. The method of claim 21, wherein (1) the elevated serum concentration is a subject concentration of the first cytokine that is greater by a predetermined third amount than a control concentration of the first cytokine; or (2) the elevated ratio is a subject ratio of a subject IL-6 concentration to a subject concentration of the second cytokine that is greater by a predetermined fourth amount than a control ratio of a control IL-6 concentration to a control concentration of the second cytokine.

23. The method of claim 21, wherein (1) the first cytokine is selected from the group consisting of Fractalkine, G-CSF, IFNα2, IFNγ, IL-13, IL-15, IL-17E/IL-25, IL-18, IL-1RA, IL-22, IL-27, IL-4, IL-5, IL-6, IL-7, IP-10, M-CSF, MCP-3, MIG, PDGF-AA, PDGF-AB/BB, TGFα, TNFα, and VEGF-A or (2) the second cytokine is selected from the group consisting of IFNα2, IFNγ, TNF, IL-17A, and IL-10.

24. (canceled)

25. (canceled)

26-79. (canceled)
